(19) United States
(12) Patent Application Publication
(10) Pub. No.: US 2026/0345462 A1
(43) Pub. Date: Nov. 5, 2026

(54) VITAMIN COMPOSITION FOR USE IN COSMETIC FORMULATIONS

(71) Applicant: Clariant International Ltd, Muttenz (CH)

(72) Inventors: Samares Chandra BISWAS, Karnataka (IN); Subramanian KALIAPPAN, Maharashtra (IN); Jayavant Ratan SHIRKE, Maharashtra (IN); Siddheshwar Bhaskar JAGTAP, Maharashtra (IN); Jesús PITARCH LÓPEZ, Frankfurt am Main (DE)

(73) Assignee: Clariant International Ltd, Muttenz (CH)

(21) Appl. No.: 19/168,354

(22) PCT Filed: Jun. 27, 2024
(86) PCT No.: PCT/EP2024/068191

§ 371 Date: Sep. 24, 2025

(30) Foreign Application Priority Data
Jun. 30, 2023 (IN) 202321043945
Nov. 24, 2023 (EP) 23212013.9

(51) Int. Cl. A61K 8/67; A61K 8/42; A61K 8/55; A61K 8/86; A61K 9/08; A61K 31/375; A61K 31/455; A61K 47/10; A61K 47/18; A61K 47/24; A61Q 19/00

(52) U.S. Cl. CPC A61K 8/676; A61K 8/42; A61K 8/553; A61K 8/675; A61K 8/86; A61K 9/08; A61K 31/375; A61K 31/455; A61K 47/10; A61K 47/18; A61K 47/24; A61Q 19/00

(57) ABSTRACT

The present invention relates to a vitamin composition comprising
(a) a vitamin or derivative thereof;
(b) a lipid component;
(c) optionally an emulsifier;
(d) a solvent; and
(e) optionally urea or a derivative thereof.

The present invention relates to a vitamin composition, a process for the preparation of the vitamin composition, various uses of the vitamin composition, as well as a cosmetic formulation comprising the vitamin composition. The present invention also relates to the use of certain compounds for vitamin stabilization.

Vitamins are multifunctional substances that are frequently used in cosmetic products. They are, for example, used as antioxidants, anti-ageing agents, skin lightening agents, or anti-inflammatory agents. Furthermore, vitamins are, for example, used to strengthen the skin barrier or to protect skin or hair from oxidative stress or damage, including UV-induced oxidative stress or damage.

There are two main groups of vitamins—fat-soluble vitamins and water-soluble vitamins. The fat-soluble vitamins include vitamins A, D, E, and K. The water-soluble vitamins include vitamin C and the B vitamins.

Water-soluble vitamins are unstable under certain conditions. They can be easily oxidized or decomposed by light, oxygen, high temperatures, alkali, metals, and/or at high or low pH, and/or by interaction with certain ingredients present in cosmetic products. For instance, ascorbic acid will oxidize fast in aqueous solutions. Niacinamide, for instance, will hydrolyze at low pH values. Maintaining the stability of vitamin C and the B vitamins, including niacinamide, in aqueous medium remains a challenge.

There is an ongoing need for vitamin-delivery systems that can be used in cosmetic products. In particular, there is a need for vitamin-delivery systems that are stable as such and that lead to cosmetic products with a stable vitamin content when incorporated thereinto. There is a particular need for vitamin-delivery systems that are stable in aqueous medium.

It has now been found that a combination of certain ingredients can be used to stabilize vitamins or derivatives thereof.

Accordingly, the present invention relates to a vitamin composition comprising
(a) a vitamin or derivative thereof;
(b) a lipid component;
(c) optionally an emulsifier;
(d) a solvent; and
(e) optionally urea or a derivative thereof.

In preferred embodiments, the vitamin composition further comprises water.

Advantageously, the vitamin composition of the present invention is stable, in particular storage-stable. The vitamin composition can be incorporated into a cosmetic formulation, for example a skin serum. Advantageously, incorporation of the vitamin composition into a cosmetic formulation provides a cosmetic composition with a stable vitamin content, even at a relatively low pH.

Advantageously, the vitamin composition of the present invention shows a slower release profile, which is useful for a variety of applications, for example where the vitamin shall be delivered onto and/or into the skin or hair over time.

Advantageously, the vitamin composition of the present invention is easy to prepare, easy to handle, and easy to use in a cosmetic formulation.

The vitamin composition of the present invention comprises (a) a vitamin or derivative thereof.

Preferably, the vitamin is selected from vitamin A, vitamin B, vitamin C, vitamin D, vitamin E, vitamin K, and mixtures thereof. More preferably, the vitamin is selected from vitamin B, vitamin C, and mixtures thereof. Particularly preferably, the vitamin is vitamin B. Also particularly preferably, the vitamin is vitamin C.

In a preferred embodiment, the vitamin or derivative thereof is selected from ascorbic acid, sodium ascorbyl phosphate, magnesium ascorbyl phosphate, ascorbyl glucoside, tetrahexyldecyl ascorbate, ethylated ascorbic acid, 3-O-glycosyl-ascorbic acid, ascorbic acid 2-glucoside, and mixtures thereof.

In a preferred embodiment, the vitamin or derivative thereof is selected from niacin (nicotinic acid), niacinamide (nicotinamide), nicotinamide mononucleotide, nicotinamide riboside, dihydronicotinic acid riboside, 1-methylnicotinamide, inositol hexaniacinate, and mixtures thereof.

In a preferred embodiment, the vitamin or derivative thereof is selected from ascorbic acid, niacinamide, and mixtures thereof. In a particularly preferred embodiment, the vitamin or derivative thereof is ascorbic acid. In a particularly preferred embodiment, the vitamin or derivative thereof is niacinamide.

In a preferred embodiment, the vitamin is selected from ascorbic acid, niacinamide, and mixtures thereof. In a particularly preferred embodiment, the vitamin is ascorbic acid. In a particularly preferred embodiment, the vitamin is niacinamide.

The vitamin composition of the present invention comprises (b) a lipid component.

Preferably, the lipid component is selected from lipids, phospholipids, and mixtures thereof. More preferably, the lipid component is selected from phospholipids.

Preferably, the lipids are selected from fatty acid esters. More preferably, the lipids are selected from triglycerides of one or more fatty acids, esters of one or more fatty acids with one or more fatty alcohols, and mixtures thereof. Even more preferably, the lipids are selected from triglycerides of one or more fatty acids having 8 to 24 carbon atoms, esters of one or more fatty acids having 8 to 24 carbon atoms with one or more fatty alcohols having 8 to 24 carbon atoms, and mixtures thereof.

Preferably, the phospholipids are selected from phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, ceramide phosphorylcholine, ceramide phosphorylethanolamine, ceramide phosphoryllipid, and mixtures thereof. Particularly preferably, the phospholipid is phosphatidylcholine.

Optionally, the vitamin composition of the present invention comprises (c) an emulsifier. In a preferred embodiment, the vitamin composition comprises an emulsifier.

In a preferred embodiment, the emulsifier is selected from fatty alcohols, fatty acids, glyceryl esters, sorbitan esters, sorbitol esters, polyglyceryl esters, sucrose esters, phosphates, fatty alcohol ethoxylates, fatty acid ethoxylates, lactylates, and mixtures thereof.

In a more preferred embodiment, the emulsifier is selected from fatty alcohols having 8 to 22 carbon atoms, fatty acids having 8 to 22 carbon atoms, esters of glycerol based on one or more C8-C22 fatty acids, esters of sorbitan based on one or more C8-C22 fatty acids, esters of sorbitol based on one or more C8-C22 fatty acids, esters of polyglycerol based on one or more C8-C22 fatty acids, esters of sucrose based on one or more C8-C22 fatty acids, esters of phosphoric acid based on one or more C8-C22 fatty alcohols, esters of phosphoric acid based on one or more ethoxylated C8-C22 fatty alcohols, C8-C22 fatty alcohol ethoxylates, C8-C22 fatty acid ethoxylates, esters of lactic acid based on a C8-C22 fatty acid, and mixtures thereof.

In an even more preferred embodiment, the emulsifier is selected from fatty alcohols having 16 to 20 carbon atoms, fatty acids having 16 to 20 carbon atoms, esters of glycerol based on one or more C16-C20 fatty acids, esters of sorbitan based on one or more C16-C20 fatty acids, esters of sorbitol based on one or more C16-C20 fatty acids, esters of polyglycerol based on one or more C16-C20 fatty acids, esters of sucrose based on one or more C16-C20 fatty acids, esters of phosphoric acid based on one or more C16-C20 fatty alcohols, esters of phosphoric acid based on one or more ethoxylated C16-C20 fatty alcohols, C16-C20 fatty alcohol ethoxylates, C16-C20 fatty acid ethoxylates, esters of lactic acid based on a C16-C20 fatty acid, and mixtures thereof.

In a particularly preferred embodiment, the emulsifier is selected from fatty alcohols having 16 to 18 carbon atoms, fatty acids having 16 to 18 carbon atoms, esters of glycerol based on one or more C16-C18 fatty acids, esters of sorbitan based on one or more C16-C18 fatty acids, esters of sorbitol based on one or more C16-C18 fatty acids, esters of polyglycerol based on one or more C16-C18 fatty acids, esters of sucrose based on one or more C16-C18 fatty acids, esters of phosphoric acid based on one or more C16-C18 fatty alcohols, esters of phosphoric acid based on one or more ethoxylated C16-C18 fatty alcohols, C16-C18 fatty alcohol ethoxylates, C16-C18 fatty acid ethoxylates, esters of lactic acid based on a C16-C18 fatty acid, and mixtures thereof.

In a preferred embodiment, the emulsifier is selected from fatty alcohol ethoxylates, fatty acid ethoxylates, and mixtures thereof. In a more preferred embodiment, the emulsifier is selected from C8-C22 fatty alcohol ethoxylates, C8-C22 fatty acid ethoxylates, and mixtures thereof. In an even more preferred embodiment, the emulsifier is selected from C16-C20 fatty alcohol ethoxylates, C16-C20 fatty acid ethoxylates, and mixtures thereof. In a particularly preferred embodiment, the emulsifier is selected from C16-C18 fatty alcohol ethoxylates, C16-C18 fatty acid ethoxylates, and mixtures thereof.

Preferably, the above-mentioned fatty alcohol ethoxylates contain from 10 to 80, preferably from 20 to 60, more preferably from 30 to 50, particularly preferably from 35 to 45 ethylene oxy units, as an average, per fatty alcohol unit.

Preferably, the above-mentioned fatty acid ethoxylates contain from 10 to 80, preferably from 20 to 60, more preferably from 30 to 50, particularly preferably from 35 to 45 ethylene oxy units, as an average, per fatty acid unit.

In a preferred embodiment, the emulsifier is selected from fatty acid ethoxylates.

In a more preferred embodiment, the emulsifier is selected from C8-C22 fatty acid ethoxylates. C8-C22 as used in this context refers to the number of carbon atoms in the fatty acid unit. Preferably, the C8-C22 fatty acid ethoxylates contain from 10 to 80, preferably from 20 to 60, more preferably from 30 to 50, particularly preferably from 35 to 45 ethylene oxy units, as an average, per fatty acid unit.

In an even more preferred embodiment, the emulsifier is selected from C16-C20 fatty acid ethoxylates. C16-C20 as used in this context refers to the number of carbon atoms in the fatty acid unit. Preferably, the C16-C20 fatty acid ethoxylates contain from 10 to 80, preferably from 20 to 60, more preferably from 30 to 50, particularly preferably from 35 to 45 ethylene oxy units, as an average, per fatty acid unit.

In a particularly preferred embodiment, the emulsifier is selected from C16-C18 fatty acid ethoxylates. C16-C18 as used in this context refers to the number of carbon atoms in the fatty acid unit. Preferably, the C16-C18 fatty acid ethoxylates contain from 10 to 80, preferably from 20 to 60, more preferably from 30 to 50, particularly preferably from 35 to 45 ethylene oxy units, as an average, per fatty acid unit.

Particularly preferably, the emulsifier is PEG-40 Stearate, herein also referred to as PEG-40 Monostearate.

The vitamin composition of the present invention comprises (d) a solvent.

In a preferred embodiment, the solvent is selected from propylene glycol (1,2-propanediol), 1,3-propanediol, 1,2-butanediol, 1,3-butanediol, 1,2-pentanediol, dipropylene glycol, ethylene glycol, polyethylene glycol, sorbitol, glycerin, and mixtures thereof. In a more preferred embodiment, the solvent is selected from propylene glycol, 1,3-propanediol, and mixtures thereof. In a particularly preferred embodiment, the solvent is propylene glycol.

Optionally, the vitamin composition of the present invention comprises (e) urea or a derivative thereof. In a preferred embodiment, the vitamin composition comprises urea or a derivative thereof.

In a preferred embodiment, the urea derivative is selected from 2-hydroxyethyl urea, 2-hydroxymethyl urea, bis(hydroxyethyl) urea, bis(hydroxymethyl) urea, N-(2-hydroxyethyl)ethylene urea, and mixtures thereof. In a more preferred embodiment, the urea derivative is selected from 2-hydroxyethyl urea, 2-hydroxymethyl urea, and mixtures thereof. In a particularly preferred embodiment, the urea derivative is 2-hydroxyethyl urea. The term “urea derivative” as used herein refers to a derivative of urea.

Optionally, the vitamin composition of the present invention further comprises (f) water.

In a preferred embodiment, the vitamin composition comprises water. In another embodiment, the vitamin composition does not contain water.

In preferred embodiments, the vitamin composition of the present invention comprises
(a) a vitamin or derivative thereof;
(b) a lipid component;
(c) an emulsifier;
(d) a solvent; and
(e) optionally urea or a derivative thereof.

In preferred embodiments, the vitamin composition of the present invention comprises
(a) a vitamin or derivative thereof;
(b) a lipid component;
(c) optionally an emulsifier;
(d) a solvent; and
(e) urea or a derivative thereof.

In preferred embodiments, the vitamin composition of the present invention comprises
(a) a vitamin or derivative thereof;
(b) a lipid component;
(c) an emulsifier;
(d) a solvent; and
(e) urea or a derivative thereof.

In preferred embodiments, the vitamin or derivative thereof is present in the vitamin composition in an amount of from 1 to 40 wt-%, preferably from 5 to 30 wt-%, more preferably from 7 to 25 wt-%, even more preferably from 8 to 20 wt-%, particularly preferably from 8 to 15 wt-%, for example from 8 to 12 wt-%, based on the total weight of the vitamin composition.

In preferred embodiments, the lipid component is present in the vitamin composition in an amount of from 0.5 to 25 wt-%, preferably from 1.0 to 20 wt-%, more preferably from 1.5 to 15 wt-%, even more preferably from 1.5 to 12 wt-%, particularly preferably from 1.5 to 5 wt-%, for example from 2 to 3 wt-%, based on the total weight of the vitamin composition.

In preferred embodiments, the emulsifier is present in the vitamin composition in an amount of from 3 to 30 wt-%, preferably from 5 to 25 wt-%, more preferably from 6 to 20 wt-%, even more preferably from 7 to 17 wt-%, particularly preferably from 10 to 15 wt-%, for example from 12 to 14 wt-%, based on the total weight of the vitamin composition.

In preferred embodiments, the emulsifier is present in the vitamin composition in an amount of from 0 to 30 wt-%, preferably from 0 to 25 wt-%, more preferably from 0 to 20 wt-%, even more preferably from 0 to 17 wt-%, particularly preferably from 0 to 15 wt-%, based on the total weight of the vitamin composition.

In preferred embodiments, the solvent is present in the vitamin composition in an amount of from 4 to 30 wt-%, preferably from 6 to 25 wt-%, more preferably from 7 to 20 wt-%, even more preferably from 8 to 17 wt-%, particularly preferably from 10 to 15 wt-%, based on the total weight of the vitamin composition.

In preferred embodiments, urea or a derivative thereof is present in the vitamin composition in an amount of from 3 to 30 wt-%, preferably from 5 to 25 wt-%, more preferably from 6 to 20 wt-%, even more preferably from 7 to 18 wt-%, particularly preferably from 10 to 15 wt-%, based on the total weight of the vitamin composition.

In preferred embodiments, urea or a derivative thereof is present in the vitamin composition in an amount of from 0 to 30 wt-%, preferably from 0 to 25 wt-%, more preferably from 0 to 20 wt-%, even more preferably from 0 to 18 wt-%, particularly preferably from 0 to 15 wt-%, based on the total weight of the vitamin composition.

In preferred embodiments, water is present in the vitamin composition in an amount of from 20 to 70 wt-%, preferably from 25 to 60 wt-%, more preferably from 30 to 55 wt-%, even more preferably from 35 to 52 wt-%, particularly preferably from 40 to 50 wt-%, based on the total weight of the vitamin composition.

In preferred embodiments, water is present in the vitamin composition in an amount of from 0 to 70 wt-%, preferably from 0 to 60 wt-%, more preferably from 0 to 55 wt-%, even more preferably from 0 to 52 wt-%, particularly preferably from 0 to 50 wt-%, based on the total weight of the vitamin composition.

In preferred embodiments, the weight ratio of (a) the vitamin or derivative thereof to (b) the lipid component is from 20:1 to 1:4, preferably from 15:1 to 1:2, more preferably from 10:1 to 1:1.5, particularly preferably from 6:1 to 3:1, for example from 5:1 to 3.5:1.

In preferred embodiments, the weight ratio of (a) the vitamin or derivative thereof to (c) the emulsifier is from 5:1 to 1:5, preferably from 4:1 to 1:3, more preferably from 3:1 to 1:2, particularly preferably from 1:1 to 1:2, for example from 1:1.0 to 1:1.5.

In preferred embodiments, the weight ratio of (a) the vitamin or derivative thereof to (d) the solvent is from 5:1 to 1:5, preferably from 3:1 to 1:3, more preferably from 2:1 to 1:2, particularly preferably from 1:1 to 1:2, for example from 1:1.0 to 1:1.5.

In preferred embodiments, the weight ratio of (a) the vitamin or derivative thereof to (e) urea or a derivative thereof is from 5:1 to 1:5, preferably from 4:1 to 1:3, more preferably from 3:1 to 1:2, particularly preferably from 1:1 to 1:2, for example from 1:1.0 to 1:1.5.

In preferred embodiments, the weight ratio of (a) the vitamin or derivative thereof to (f) water is from 1:1 to 1:20, preferably from 1:1 to 1:10, more preferably from 1:2 to 1:6, particularly preferably from 1:3 to 1:5, for example from 1:4 to 1:5.

The present invention also relates to a process for the preparation of a vitamin composition of the present invention, wherein the process comprises:
i) preparing a mixture comprising a vitamin or derivative thereof, a lipid component, optionally an emulsifier, a solvent, and optionally urea or a derivative thereof; and
ii) optionally adding water to the mixture obtained in step i).

Step i): The process of the present invention comprises i) preparing a mixture comprising a vitamin or derivative thereof, a lipid component, optionally an emulsifier, a solvent, and optionally urea or a derivative thereof.

Preferred vitamins or derivatives thereof are described further above. Preferred lipid components are described further above. Preferred emulsifiers are described further above. Preferred solvents are described further above. Preferred urea derivatives are described further above.

Preferably, the mixture comprising a vitamin or derivative thereof, a lipid component, optionally an emulsifier, a solvent, and optionally urea or a derivative thereof is prepared at a temperature in the range of from 20 to 150 °C, more preferably from 50 to 120 °C, even more preferably from 60 to 100 °C, particularly preferably from 70 to 95 °C, for example from 75 to 90 °C.

Preferably, the mixture comprising a vitamin or derivative thereof, a lipid component, optionally an emulsifier, a solvent, and optionally urea or a derivative thereof is subsequently cooled to a temperature in the range of from 0 to 70 °C, more preferably from 10 to 60 °C, even more preferably from 15 to 50 °C, particularly preferably from 20 to 45 °C, for example from 20 to 25 °C or from 40 to 50 °C.

Preferably, the mixture comprising a vitamin or derivative thereof, a lipid component, optionally an emulsifier, a solvent, and optionally urea or a derivative thereof is prepared at a pressure in the range of from 400 mbar to 400 bar, more preferably from 600 mbar to 2 bar, even more preferably from 800 to 1200 mbar, particularly preferably from 1003 to 1023 mbar, for example at a pressure of 1013 mbar.

Preferably, the mixture comprising a vitamin or derivative thereof, a lipid component, optionally an emulsifier, a solvent, and optionally urea or a derivative thereof is prepared by stirring. Any stirrer can be used. For example, an overhead stirrer can be used.
Step i) of the process of the present invention can, for example, be carried out in a batch, semi-batch, semi-continuous or continuous process.
Step ii): Optionally, the process of the present invention comprises ii) adding water to the mixture obtained in step i). In a preferred embodiment, the process of the present invention comprises ii) adding water to the mixture obtained in step i).

Preferably, water is added to the mixture obtained in step i) at a temperature in the range of from 0 to 90 °C, more preferably from 10 to 60 °C, even more preferably from 15 to 50 °C, particularly preferably from 20 to 45 °C, for example from 20 to 25 °C or from 40 to 50 °C.

Preferably, water is added to the mixture obtained in step i) at a pressure in the range of from 400 mbar to 400 bar, more preferably from 600 mbar to 2 bar, even more preferably from 800 to 1200 mbar, particularly preferably from 1003 to 1023 mbar, for example at a pressure of 1013 mbar.

Preferably, water is added to the mixture obtained in step i) under stirring. Any stirrer can be used. For example, an overhead stirrer can be used.
Step ii) of the process of the present invention can, for example, be carried out in a batch, semi-batch, semi-continuous or continuous process.

The process of the present invention can, for example, be carried out in a batch, semi-batch, semi-continuous or continuous process.

In preferred embodiments, the vitamin composition of the present invention is obtained by a process of the present invention.

The present invention also relates to a vitamin composition obtained by a process of the present invention.

The present invention also relates to the use of a mixture comprising a lipid component, optionally an emulsifier, a solvent, optionally urea or a derivative thereof, and optionally water, to stabilize a vitamin or derivative thereof or to increase the bioavailability of a vitamin or derivative thereof.

In a preferred embodiment, a mixture comprising a lipid component, optionally an emulsifier, a solvent, optionally urea or a derivative thereof, and optionally water, is used to stabilize a vitamin or derivative thereof.

In a preferred embodiment, a mixture comprising a lipid component, optionally an emulsifier, a solvent, optionally urea or a derivative thereof, and optionally water, is used to increase the bioavailability of a vitamin or derivative thereof.

In a preferred embodiment, a mixture comprising a lipid component, optionally an emulsifier, a solvent, optionally urea or a derivative thereof, and optionally water, is used to stabilize a vitamin or derivative thereof and to increase the bioavailability of a vitamin or derivative thereof.

Preferred lipid components are described further above. Preferred emulsifiers are described further above. Preferred solvents are described further above. Preferred urea derivatives are described further above. Preferred vitamins or derivatives thereof are described further above.

The present invention also relates to the use of a vitamin composition of the present invention as an antioxidant, anti-ageing agent, anti-wrinkle agent, skin lightening agent, skin whitening agent, depigmenting agent, anti-inflammatory agent, or anti-acne agent.

The present invention also relates to the use of a vitamin composition of the present invention as an antioxidant, anti-ageing agent, anti-wrinkle agent, skin lightening agent, skin whitening agent, or depigmenting agent.

The present invention also relates to the use of a vitamin composition of the present invention as an anti-ageing agent, anti-wrinkle agent, skin lightening agent, skin whitening agent, or depigmenting agent.

The present invention also relates to a vitamin composition of the present invention for use as an antioxidant, anti-ageing agent, anti-wrinkle agent, skin lightening agent, skin whitening agent, depigmenting agent, anti-inflammatory agent, or anti-acne agent.

The present invention also relates to a vitamin composition of the present invention for use as an antioxidant, anti-inflammatory agent, or anti-acne agent.

The present invention also relates to a vitamin composition of the present invention for use as an anti-inflammatory agent, or anti-acne agent.

The present invention also relates to the use of a vitamin composition of the present invention to strengthen the skin barrier, to protect skin or hair from oxidative stress or damage, including UV-induced oxidative stress or damage, to protect skin or hair against photoaging, to protect skin against UV-induced immunosuppression, to protect skin against photo carcinogenesis, to promote differentiation of keratinocytes, to induce collagen synthesis, or to inhibit melanogenesis.

The present invention also relates to the use of a vitamin composition of the present invention to strengthen the skin barrier, to protect hair from oxidative stress or damage, including UV-induced oxidative stress or damage, to protect skin or hair against photoaging, to promote differentiation of keratinocytes, to induce collagen synthesis, or to inhibit melanogenesis.

The present invention also relates to the use of a vitamin composition of the present invention to protect skin or hair against photoaging, to promote differentiation of keratinocytes, or to induce collagen synthesis.

The present invention also relates to a vitamin composition of the present invention for use in strengthening the skin barrier, protecting skin or hair from oxidative stress or damage, including UV-induced oxidative stress or damage, protecting skin or hair against photoaging, protecting skin against UV-induced immunosuppression, protecting skin against photo carcinogenesis, promoting differentiation of keratinocytes, inducing collagen synthesis, or inhibiting melanogenesis.

The present invention also relates to a vitamin composition of the present invention for use in strengthening the skin barrier, protecting skin or hair from oxidative stress or damage, including UV-induced oxidative stress or damage, protecting skin against UV-induced immunosuppression, protecting skin against photo carcinogenesis, or inhibiting melanogenesis.

The present invention also relates to a vitamin composition of the present invention for use in protecting skin from oxidative stress or damage, including UV-induced oxidative stress or damage, protecting skin against UV-induced immunosuppression, or protecting skin against photo carcinogenesis.

The present invention also relates to the use of a vitamin composition of the present invention in a cosmetic formulation.

The present invention also relates to a cosmetic formulation comprising
(A) a vitamin composition of the present invention, wherein said vitamin composition is present in the cosmetic formulation in an amount such that the amount of the vitamin or derivative thereof is from 1 to 30 wt-%, preferably from 5 to 25 wt-%, more preferably from 6 to 20 wt-%, even more preferably from 7 to 15 wt-%, particularly preferably from 8 to 12 wt-%, based on the total weight of the cosmetic formulation; and
(B) one or more further components.

Preferably, the cosmetic formulation is selected from the group consisting of skin oil, skin care cream, skin care lotion, ointment, skin conditioner, face spray, body spray, hand oil, shower bath, hair conditioner, shaving gel, shampoo, body wash, facial cleanser, face mask, bubble bath, intimate wash, bath oil, cleansing milk, micellar water, make-up remover, cleansing wipes, hair mask, perfume, liquid soap, shaving soap, shaving foam, cleansing foam, day cream, anti-ageing cream, body milk, body lotion, body mousse, skin serum, face serum, hand serum, nail serum, eye cream, sunscreen lotion, sun cream, face cream, after-shave lotion, pre-shaving cream, depilatory cream, skin-whitening gel, self-tanning cream, anti-acne gel, mascara, foundation, primer, concealer, blush, bronzer, blemish balm (bb) cream, eyeliner, night cream, eye brow gel, highlighter, lip stain, hand sanitizer, hair oil, nail varnish remover, conditioner, hair styling gel, hair styling cream, anti-frizz serum, scalp treatment, hair colorant, split end fluid, deodorant, antiperspirant, baby cream, insect repellent, hand cream, sunscreen gel, foot cream, exfoliator, face scrub, body scrub, cellulite treatment, cuticle cream, lip balm, hair treatment, eye shadow, bath additive, body mist, eau de toilette, mouthwash, toothpaste, lubricating gel, moisturizer, serum, toner, aqua sorbet, skin gel, cream gel, styling mousse, lip stick, lip gloss, body oil, shower milk, illuminator, lip crayon, hair spray, combing cream, and sunblock.

More preferably, the cosmetic formulation is selected from the group consisting of skin oil, day cream, anti-aging cream, body milk, body lotion, body mousse, skin serum, face serum, hand serum, nail serum, eye cream, sunscreen lotion, sun cream, face cream, after-shave lotion, skin-whitening gel, self-tanning cream, anti-acne gel, foundation, primer, concealer, blemish balm (bb) cream, night cream, eye brow gel, hand sanitizer, skin conditioner, deodorant, antiperspirant, baby cream, insect repellent, hand cream, sunscreen gel, foot cream, exfoliator, face scrub, body scrub, cellulite treatment, nail cuticle cream, lip balm, body mist, eau de toilette, lubricating gel, moisturizer, serum, toner, aqua sorbet, skin gel, cream gel, body oil, illuminator, and sunblock.

Even more preferably, the cosmetic formulation is selected from the group consisting of skin oil, day cream, anti-aging cream, body milk, body lotion, skin serum, face serum, hand serum, nail serum, eye cream, sunscreen lotion, face cream, night cream, baby cream, hand cream, foot cream, exfoliator, face scrub, body scrub, nail cuticle cream, lip balm, moisturizer, serum, toner, skin gel, cream gel, body oil, and sunblock.

Particularly preferably, the cosmetic formulation is selected from the group consisting of skin serum, face serum, hand serum, nail serum.

In preferred embodiments, the cosmetic formulation has a pH value in the range of from 1.5 to 8, more preferably from 2 to 6, even more preferably from 2.5 to 4, particularly preferably from 3 to 3.5.

In at least one embodiment, the formulation comprises a solvent. In at least one embodiment, the formulation comprises a solvent, wherein the solvent comprises water and/or alcohol. Solvent is useful for providing the compounds used in present invention in liquid form. In at least one embodiment, the solvent is cosmetically acceptable. In at least one embodiment, the formulation comprises at least 10 wt.-%, preferably at least 20 wt.-%, more preferably at least 30 wt.-%, even more preferably at least 50 wt.-% water. Water is useful for economic reasons but also because it is cosmetically acceptable. Optionally, the formulation comprises water-miscible or water-soluble solvents, such as lower alkyl alcohols. In at least one embodiment, the formulation comprises C1-C5 alkyl monohydric alcohols, preferably C2-C3 alkyl monohydric alcohols. The alcohols which may be present are in particular lower monohydric or polyhydric alcohols having 1 to 4 carbon atoms customarily used for cosmetic purposes, such as preferably ethanol and isopropanol.

In a preferred embodiment, the formulation comprises a solvent selected from the group consisting of water, glycols, ethanol, and mixtures thereof. In a preferred embodiment, the formulation comprises water. In one embodiment, the formulation is an aqueous solution.

In a preferred embodiment, the formulation comprises an aqueous, alcoholic or aqueous-alcoholic solvent, wherein the aqueous, alcoholic or aqueous-alcoholic solvent comprises water, ethanol, propanol, isopropanol, 1,2-propylene glycol, 1,3-propylene glycol, isobutanol, butanol, butyl glycol, butyl diglycol, 1,2-pentanediol, dipropylene glycol, glycerol, or mixtures thereof; preferably wherein the aqueous, alcoholic or aqueous-alcoholic solvent comprises water, ethanol, propanol, isopropanol, 1,2-propylene glycol, 1,3-propylene glycol, glycerol, or mixtures thereof; more preferably wherein the aqueous, alcoholic or aqueous-alcoholic solvent comprises water, isopropanol, 1,2-propylene glycol, 1,3-propylene glycol, or mixtures thereof; even more preferably wherein the aqueous, alcoholic or aqueous-alcoholic solvent consists of water or consists of a mixture of water and an alcohol wherein the alcohol is selected from the group consisting of isopropanol, 1,2-propylene glycol and 1,3-propylene glycol.

In at least one embodiment, the formulation comprises additives common in cosmetology, pharmacy, and dermatology, which are hereinafter called auxiliaries. In at least one embodiment, the auxiliary is selected from the group consisting of oily substances, cationic polymers, film formers, superfatting agents, stabilizers, active biogenic substances, glycerol, preservatives, pearlizing agents, dyes and fragrances, solvents, opacifiers, functional acids, and also protein derivatives such as gelatin, collagen hydrolysates, natural or synthetic-based polypeptides, egg yolk, lecithin, lanolin and lanolin derivatives, fatty alcohols, silicones, deodorants, substances with a keratolytic and keratoplastic action, enzymes, and/or carriers/solvents.

In at least one embodiment, the formulation comprises water soluble vitamins and their derivatives, water soluble amino acids and their salts and/or derivatives, viscosity modifiers, dyes, nonvolatile solvents or diluents (water soluble and insoluble), pearlescent aids, thickeners, foam boosters, surfactants or co-surfactants, pediculocides, pH adjusting agents, perfumes, preservatives, chelants, proteins, skin active agents, sunscreens, UV absorbers, vitamins, caffeine, minoxidil, and combinations thereof. In at least one embodiment, the formulation comprises from 0 wt.-% to 5 wt.-% vitamins and amino acids, by total weight of the formulation.

In at least one embodiment, the formulation comprises an oily substance, which is any fatty substance which is liquid at room temperature (25 °C). In at least one embodiment, the formulation comprises oily substance selected from the group consisting of silicone oils, volatile or nonvolatile, linear, branched or cyclic, optionally with organic modification; phenylsilicones; silicone resins and silicone gums; mineral oils such as paraffin oil or vaseline oil; oils of animal origin such as perhydrosqualene, lanolin; oils of plant origin such as liquid triglycerides, e.g., sunflower oil, corn oil, soybean oil, rice oil, jojoba oil, babusscu oil, pumpkin oil, grapeseed oil, sesame oil, walnut oil, apricot oil, macadamia oil, avocado oil, sweet almond oil, lady's-smock oil, castor oil, triglycerides of caprylic/capric acids, olive oil, peanut oil, rapeseed oil, argan oil, abyssinian oil, and coconut oil; synthetic oils such as purcellin oil, isoparaffins, linear and/or branched fatty alcohols and fatty acid esters, preferably guerbet alcohols having 6 to 18, preferably 8 to 10, carbon atoms; esters of linear (C6-C13) fatty acids with linear (C6-C20) fatty alcohols; esters of branched (C6-C13) carboxylic acids with linear (C6-C20) fatty alcohols, esters of linear (C6-C18) fatty acids with branched alcohols, especially 2-ethylhexanol; esters of linear and/or branched fatty acids with polyhydric alcohols (such as dimerdiol or trimerdiol, for example) and/or guerbet alcohols; triglycerides based on (C6-C10) fatty acids; esters such as dioctyl adipate, diisopropyl dimer dilinoleate; propylene glycols/dicaprylate or waxes such as beeswax, paraffin wax or microwaxes, alone or in combination with hydrophilic waxes, such as cetylstearyl alcohol, for example; fluorinated and perfluorinated oils; fluorinated silicone oils; mixtures of the aforementioned compounds.

In at least one embodiment, the formulation comprises a cationic polymer. Suitable cationic polymers include those known under the INCI designation “Polyquaternium”, especially Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, and also Polyquaternium 37 & mineral oil & PPG trideceth (Salcare SC95), PVP dimethylaminoethyl methacrylate copolymer, guar-hydroxypropyltriammonium chlorides, and also calcium alginate and ammonium alginate. It is additionally possible to employ cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as amidomethicones, for example; copolymers of adipic acid and dimethylaminohydroxypropyldiethylenetriamine; polyaminopolyamide and cationic chitin derivatives, such as chitosan, for example.

In at least one embodiment, the formulation comprises a superfatting agent. As superfatting agents it is possible to use substances such as, for example, polyethoxylated lanolin derivatives, lecithin derivatives, polyol fatty acid esters, monoglycerides, or fatty acid alkanol amides, the latter serving simultaneously as foam stabilizers. Moisturizers available include for example isopropyl palmitate, glycerol and/or sorbitol.

In at least one embodiment, the formulation comprises a stabilizer. As stabilizer it is possible to use metal salts of fatty acids, such as magnesium, aluminum and/or zinc stearate, for example.

In at least one embodiment, the formulation comprises a care additive. The formulations can be blended with conventional ceramides, pseudoceramides, cholesterol, cholesterol fatty acid esters, cerebrosides, phospholipids, panthenol and similar substances as a care additive.

In at least one embodiment, the formulation comprises a preservative or preservative system. Examples of suitable preservatives include benzyl alcohol, piroctone olamine, phenoxyethanol, parabens, pentanediol, benzoic acid/sodium benzoate, sorbic acid/potassium sorbate, caprylhydroxamic acid, or combinations thereof. Examples of suitable preservation boosting ingredients include anisic acid, lactic acid, sorbitan caprylate, ethylhexylglycerin, methylheptylglycerin, caprylyl glycol, caprylyl glyceryl ether, octanediol, Capryloyl/Caproyl Anhydro Methyl Glucamide (Velsan® Flex), or combinations thereof. In at least one embodiment, the formulation comprises from 0.01 to 5 wt.-%, particularly preferably from 0.05 to 1 wt.-% of at least one preservative.

In at least one embodiment, the formulation comprises a preservative selected from the group consisting of cetyltrimethyl ammonium chloride, cetylpyridinium chloride, benzethonium chloride, diisobutylethoxyethyldimethyl benzylammonium chloride, sodium N-lauryl sarcosinate, sodium N-palmethylsarcosinate, lauroylsarcosine, N-myristoylglycine, potassium-N-laurylsarcosine, trimethylammonium chloride, sodium aluminium chlorohydroxylactate, triethylcitrate, tricetylmethylammonium chloride, 2,4,4′-trichloro-2′-hydroxydiphenylether (Triclosan), phenoxyethanol, 1,5-pentanediol, 1,6-hexanediol, 3,4,4′-trichlorocarbanilide (Triclocarban), diaminoalkylamide, L-lysine hexadecylamide, heavy metal citrate salts, salicylate, piroctone, zinc salts, pyrithione and its heavy metal salts, zinc pyrithione, zinc phenol sulfate, farnesol, ketoconazol, oxiconazol, bifonazole, butoconazole, cloconazole, clotrimazole, econazole, enilconazole, fenticonazole, isoconazole, miconazole, sulconazole, tioconazole, fluconazole, itraconazole, terconazole, naftifine, terbinafine, selenium disulfide, methylchloroisothiazolinone, methylisothiazolinone, methyldibromo glutaronitrile, piroctone olamine (Octopirox®), AgCl, chloroxylenol, sodium salts of diethylhexylsulfosuccinate, sodium benzoate, phenoxyethanol, benzyl alcohol, phenoxyisopropanol, paraben, such as butyl-, ethyl-, methyl- and propylparaben, and their salts, pentanediol, 1,2-octanediol, ethylhexylglycerin, benzyl alcohol, sorbic acid, benzoic acid, lactic acid, imidazolidinyl urea, diazolidinyl urea, dimethylol dimethyl hydantoin (DMDMH), sodium salts of hydroxymethyl glycinate, hydroxyethylglycine of sorbic acid, and combinations thereof. In at least one embodiment, the preservative is selected from the group consisting of phenoxyethanol, benzyl paraben, butyl paraben, ethyl paraben, isobutyl paraben, isopropyl paraben, methyl paraben, propyl paraben, iodopropynyl butylcarbamate, methyldibromoglutaronitrile, DMDM hydantoin, and combinations thereof. In at least one embodiment, the formulation is substantially free of parabens.

In at least one embodiment, the formulation comprises an anti-fungal substance. In at least one embodiment, the anti-fungal substance is selected from the group consisting of ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, zinc pyrithione, piroctone olamine (octopirox), and combinations thereof. In at least one embodiment, the formulation comprises a total amount of anti-fungal substance in the formulation of from 0.1 wt.-% to 1 wt.-%. In at least one embodiment, the formulation comprises pyridinethione anti-dandruff particulates. For example, 1-hydroxy-2-pyridinethione salts are highly preferred particulate anti-dandruff agents. The concentration of pyridinethione antidandruff particulate may range from 0.1 wt.-% to 4 wt.-%, by total weight of the formulation, preferably from 0.1 wt.-% to 3 wt.-%, more preferably from 0.3 wt.-% to 2 wt.-%. Preferred pyridinethione salts include those formed from heavy metals such as zinc, tin, cadmium, magnesium, aluminum or zirconium, preferably zinc, more preferably the zinc salt of 1-hydroxy-2-pyridinethione (known as “zinc pyridinethione” or “ZPT”), more preferably 1-hydroxy-2-pyridinethione salts in platelet particle form. Salts formed from other cations, such as sodium, may also be suitable.

Functional acids are acidic substances used to impart a clinical functionality to the skin or hair upon application. Suitable functional acids include alpha-hydroxy acids, beta-hydroxy acids, lactic acid, retinoic acid, and similar substances.

In at least one embodiment, the formulation comprises an astringent. In at least one embodiment, the astringent is selected from the group consisting of magnesium oxide, aluminum oxide, titanium dioxide, zirconium dioxide, zinc oxide, oxide hydrates, aluminum oxide hydrate (boehmite) and hydroxide, chlorohydrates of calcium, magnesium, aluminum, titanium, zirconium or zinc. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% astringent.

In at least one embodiment, the formulation comprises a deodorizing agent. In at least one embodiment, the deodorizing agent is selected from the group consisting of allantoin, bisabolol, and combinations thereof. In at least one embodiment, the formulation comprises from 0.001 wt.-% to 10 wt.-%, or from 0.01 wt.-% to 9 wt.-%, or from 0.05 wt.-% to 8 wt.-%, or from 0.1 wt.-% to 5 wt.-% deodorizing agent.

In at least one embodiment, the formulation comprises a sun protection agent and/or UV filter. Suitable sun protection agents and UV filters are disclosed in WO-2013/017262A1, from page 32, line 11 to the end of page 33. In at least one embodiment, the sun protection agent and/or UV filter is selected from the group consisting of 4-amino benzoic acid, 3-(4′-trimethylammonium)-benzylide-boran-2-one-methylsulfate, camphor benzalkonium methosulfate, 3,3,5-trimethyl-cyclohexylsalicylate, 2-hydroxy-4-methoxybenzophenone, 2-phenylbenzimidazole-5-sulfonic acid and potassium-, sodium-und triethanolamine salts thereof, 3,3′-(1,4-phenylene dimethine)-bis-(7,7-dimethyl-2-oxobicyclo[2.2.1]-heptane-1-methane sulfonic acid) and its salts, 1-(4-tert.-butylphenyl)-3-(4-methoxyphenyl)propan-1,3-dion, 3-(4′-sulfo)-benzylidene-bornane-2-one its salts, 2-cyan-3,3-diphenyl-acrylic acid-(2-ethylhexylester), polymers of N-[2(and 4)-(2-oxoborn-3-ylidenmethyl)benzyl]-acrylamide, 4-methoxy-cinnamic acid-2-ethyl-hexylester, ethoxylated ethyl-4-amino-benzoate, 4-methoxy-cinnamic acid-isoamylester, 2,4,6-tris-[p-(2-ethylhexyloxycarbonyl)anilino]-1,3,5-triazine, 2-(2H-benzotriazole-2-yl)-4-methyl-6-(2-methyl-3-(1,3,3,3-tetramethyl-1-(trimethylsilyloxy)-disiloxanyl)-propyl)phenol, 4,4′-[(6-[4-((1,1-dimethylethyl)-amino-carbonyl)phenylamino]-1,3,5-triazin-2,4-yl)diimino]bis-(benzoic acid-2-ethylhexylester), 3-benzophenone, 4-benzophenone (acid), 3(4′-methylbenzyliden)-D,L-camphor, 3-benzylidene-camphor, salicylic acid-2-ethylhexylester, 4-dimethyl aminobenzic acid-2-ethylhexylester, hydroxy-4-methoxy-benzophenone-5 sulfonic acid and the sodium salt thereof, 4-isopropyl benzylsalicylate, N,N,N-trimethyl-4-(2-oxoborn-3-ylidenemethyl) anilium methyl sulfate, homosalate (INN), oxybenzone (INN), 2-phenylbenzimidazole-5-sulfonic acid and its sodium, potassium, and triethanolamine salts, octylmethoxy cinnamic acid, isopentyl-4-methoxy cinnamic acid, isoamyl-p-methoxy cinnamic acid, 2,4,6-trianilino-(p-carbo-2′-ethylhexyl-1′-oxy)-1,3,5-triazine (octyl triazone) phenol, 2,2(2H-benzotriazol-2-yl)-4-methyl-6-(2-methyl-3-(1,3,3,3-tetramethyl-1-(trimethylsilyl)oxy)-disiloxanyl)propyl (drometrizole trisiloxane) benzic acid, 4,4-((6-(((1,1-dimethylethyl)amino)carbonyl)phenyl)amino)-1,3,5-triazine-2,4-diyl)diimino)bis,bis(2-ethylhexyl)ester) benzoic acid, 4,4-((6-(((1,1-dimethylethyl)amino)-carbonyl)phenyl)amino)-1,3,5-triazine-2,4-diyl)diimino)bis,bis(2-ethylhexyl)ester), 3-(4′-methylbenzylidene)-D,L-camphor (4-methylbenzylidene camphor), benzylidene-camphor-sulfonic acid, octocrylene, polyacrylamidomethyl-benzylidene-camphor, 2-ethylhexyl salicylate (octyl salicylate), 4-dimethyl-aminobenzoeacidethyl-2-hexylester (octyl dimethyl PABA), PEG-25 PABA, 2 hydroxy-4-methoxybenzo-phenone-5-sulfonic acid (5-benzophenone) and the sodium salt thereof, 2,2′-methylene-bis-6-(2H-benzotriazol-2-yl)-4-(tetramethyl-butyl)-1,1,3,3-phenol, the sodium salt of 2-2′-bis-(1,4-phenylene)1H-benzimidazole-4,6-disulfonic acid, (1,3,5)-triazine-2,4-bis((4-(2-ethyl-hexyloxy)-2-hydroxy)-phenyl)-6-(4-methoxyphenyl), 2-ethylhexyl-2-cyano-3,3-diphenyl-2-propenoate, glyceryl octanoate, di-p-methoxy cinnamic acid, p-amino-benzoic acid and its ester, 4-tert-butyl-4′-methoxydibenzoylmethane, 4-(2-p-glucopyranoxy)propoxy-2-hydroxybenzophenone, octyl salicylate, methyl-2,5-diisopropyl cinnamic acid, cinoxate, dihydroxy-dimethoxybenzophenone, disodium salts of 2,2′-dihydroxy-4,4′-dimethoxy-5,5′-disulfobenzophenone, dihydroxybenzophenone, 1,3,4-dimethoxyphenyl-4,4-dimethyl-1,3-pentanedione, 2-ethylhexyl-dimethoxybenzyliden-dioxoimidazolidinpropionate, methylene-bis-benztriazolyl tetramethylbutylphenol, phenyldibenzimidazoltetrasulfonate, bis-ethylhexyloxyphenol-methoxyphenol-triazine, tetrahydroxybenzophenone, terephthalylidendicamphor-sulfonic acid, 2,4,6-tris[4,2-ethylhexyloxycarbonyl)anilino]-1,3,5-triazine, methyl-bis(trimethylsiloxy)silyl-isopentyl trimethoxy cinnamic acid, amyl-p-dimethylaminobenzoate, amyl-p-dimethylamino benzoate, 2-ethylhexyl-p-dimethylaminobenzoate, isopropyl-p-methoxy cinnamic acid/diisopropyl cinnamic acid ester, 2-ethylhexyl-p-methoxy cinnamic acid, 2-hydroxy-4-methoxy benzophenone, 2-hydroxy-4-methoxybenzophenone-5-sulfonic acid and the trihydrate, 2-hydroxy-4-methoxybenzophenone-5-sulfonate sodium salt, phenyl-benzimidazole sulfonic acid, and combinations thereof. In at least one embodiment, the formulation comprises from 0.001 wt.-% to 10 wt.-%, preferably from 0.05 wt.-% to 5 wt.-%, even more preferably from 0.1 wt.-% to 3 wt.-%, most preferably from 0.05 wt.-% to 1 wt.-% sun protection agent and/or UV filter. In at least one embodiment, the formulation comprises a photoprotective substance in an amount of from 0.01 to 10 wt.-%, or from 0.1 to 5 wt.-%, more preferably from 0.2 to 2 wt.-%. Suitable photoprotective substances include, in particular, all of the photoprotective substances specified in EP1084696A1, which is incorporated herein by reference. In a preferred embodiment, the photoprotective substance is selected from the group consisting of 2-ethylhexyl 4-methoxycinnamate, methyl methoxycinnamate, 2-hydroxy-4-methoxybenzophenone-5-sulfonic acid, polyethoxylated p-aminobenzoates, and combinations thereof.

In at least one embodiment, the formulation comprises an anti-oxidant. In at least one embodiment, the anti-oxidant is selected from the group consisting of amino acids, peptides, sugars, imidazoles, carotinoids, carotenes, chlorogenic acid, lipoic acid, thiols, thiol glycosyl esters, thiol N-acetyl esters, thiol methyl esters, thiol ethyl esters, thiol propyl esters, thiol amyl esters, thiol butyl esters, thiol lauryl esters, thiol palmitoyl esters, thiol oleyl esters, thiol linoleyl esters, thiol cholesteryl esters, thiol glyceryl esters, dilaurylthiodipropionate, distearylthiodipropionate, thiodipropionic acid, metal chelators, hydroxy acids, fatty acids, folic acids, vitamin C, tocopherol, vitamin A, stilbenes, derivatives and combinations thereof. In at least one embodiment, the anti-oxidant is selected from the group consisting of glycine, histidine, tyrosine, tryptophan, urocaninic acid, D,L-carnosine, D-carnosine, L-carnosine, beta-carotene, alpha-carotene, lycopene, dihydrolipoic acid, aurothioglucose, propylthiouracil, thioredoxine, glutathione, cysteine, cystine, cystamine, buthioninsulfoximine, homocysteinsulfoximine, buthioninsulfone, penta-, hexa-, heptathioninsulfoximine, hydroxyfatty acids, palmitic acid, phytinic acid, lactoferrin, citric acid, lactic acid, malic acid, humic acid, bile acid, bilirubin, biliverdin, EDTA, EGTA, linoleic acid, linolenic acid, oleic acid, butylhydroxyanisol, trihydroxybutyrophenone, ubichinon, ubichinol, ascorbylpalmitate, Mg-ascorbylphosphate, ascorbylacetate, vitamin E acetate, vitamin A palmitate, carnosine, mannose, ZnO, ZnSO4, selenium methionine, stilbenes, superoxide dismutase, and combinations thereof. In at least one embodiment, the antioxidant is selected from the group consisting of vitamin A, vitamin A derivatives, vitamin E, vitamin E derivatives, and combinations thereof. In at least one embodiment, the formulation comprises from 0.001 wt.-% to 10 wt.-%, preferably from 0.05 wt.-% to 5 wt.-%, particularly preferably from 0.1 wt.-% to 3 wt.-%, also particularly preferably from 0.05 wt.-% to 1 wt.-% antioxidant.

In at least one embodiment, the formulation comprises a dye or pigment. In at least one embodiment, the formulation comprises at least one pigment. Suitable dyes and pigments are disclosed in WO2013/017262A1 in the table spanning pages 36 to 43. These may be colored pigments which impart color effects to the product mass or to hair, or they may be luster effect pigments which impart luster effects to the product mass or to hair. The color or luster effects on hair are preferably temporary, i.e. they last until the next hair wash and can be removed again by washing the hair with customary shampoos. In at least one embodiment, the formulation comprises a total amount of from 0.01 wt.-% to 25 wt.-%, preferably from 5 wt.-% to 15 wt.-% pigment. In at least one embodiment, the particle size of the pigment is from 1 micron to 200 micron, preferably from 3 micron to 150 micron, more preferably from 10 micron to 100 micron. The pigments are colorants which are virtually insoluble in the application medium, and may be inorganic or organic. Inorganic-organic mixed pigments are also possible. Preference is given to inorganic pigments. The advantage of inorganic pigments is their excellent resistance to light, weather and temperature. The inorganic pigments may be of natural origin. In at least one embodiment, the inorganic pigment is selected from the group consisting of chalk, ochre, umber, green earth, burnt sienna, graphite, and combinations thereof. The pigments may be white pigments, such as, for example, titanium dioxide or zinc oxide, black pigments, such as, for example, iron oxide black, colored pigments, such as, for example, ultramarine or iron oxide red, luster pigments, metal effect pigments, pearlescent pigments, and fluorescent or phosphorescent pigments, where preferably at least one pigment is a colored, nonwhite pigment. In at least one embodiment, the pigment is selected from the group consisting of metal oxides, hydroxides and oxide hydrates, mixed phase pigments, sulfur-containing silicates, metal sulfides, complex metal cyanides, metal sulfates, chromates and molybdates, and the metals themselves (bronze pigments), and combinations thereof. In at least one embodiment, the pigment is selected from the group consisting of titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), Prussian blue (ferric ferrocyanide, CI 77510), carmine (cochineal), and combinations thereof. In at least one embodiment, the pigment is selected from the group consisting of pearlescent and colored pigments based on mica which are coated with a metal oxide or a metal oxychloride, such as titanium dioxide or bismuth oxychloride, and optionally further color-imparting substances, such as iron oxides, Prussian blue, ultramarine, carmine etc. and where the color can be determined by varying the layer thickness. Such pigments are sold, for example, under the trade names Rona®, Colorona®, Dichrona® and Timiron® by Merck. In at least one embodiment, the pigment is selected from the group consisting of organic pigments such as sepia, gamboge, bone charcoal, Cassel brown, indigo, chlorophyll and other plant pigments. In at least one embodiment, the pigment is selected from the group consisting of synthetic organic pigments such as azo pigments, anthraquinoids, indigoids, dioxazine, quinacridone, phthalocyanine, isoindolinone, perylene and perinone, metal complex, alkali blue and diketopyrrolopyrrole pigments.

In at least one embodiment, the formulation comprises from 0.01 wt.-% to 10 wt.-%, preferably from 0.05 wt.-% to 5 wt.-%, of at least one particulate substance. Suitable substances are, for example, substances which are solid at room temperature (25 °C) and are in the form of particles. In at least one embodiment, the particulate substance is selected from the group consisting of silica, silicates, aluminates, clay earths, mica, insoluble salts, in particular insoluble inorganic metal salts, metal oxides, e.g. titanium dioxide, minerals and insoluble polymer particles are suitable. The particles may be present in the formulation in undissolved, preferably stably dispersed form, and, following application to the keratin substrate and evaporation of the solvent, can deposit on the substrate in solid form. A stable dispersion can be achieved by providing the formulation with a yield point which is large enough to prevent the solid particles from sinking. An adequate yield point can be established using suitable gel formers in a suitable amount. In at least one embodiment, the particulate substance is selected from the group consisting of silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

In at least one embodiment, the formulation comprises a direct dye. Preferred among the direct dyes are the following compounds, alone or in combination with one another: Hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydroxyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof. Particularly preferred among the aforesaid direct dyes are the following compounds, alone or in combination with one another: hydroxyethyl-2-nitro-p-toluidine, 2-hydroxyethylpicramic acid, 4-nitrophenylaminourea, tri(4-amino-3-methylphenyl)carbenium chloride (Basic Violet 2), 1,4-di-amino-9,10-anthracenedione (Disperse Violet 1), 1-(2-hydroxy-ethyl)amino-2-nitro-4-[di(2-hydro-xyethyl)amino]benzene (HC Blue No. 2), 4-[ethyl-(2-hydroxyethyl)amino]-1-[(2-hydroxyethyl)amino]-2-nitrobenzene hydrochloride (HC Blue No. 12), 1-amino-4-[di(2-hydroxyethyl)amino]-2-nitrobenzene hydrochloride (HC Red No. 13), 4-amino-1-[(2-hydroxyethyl)amino]-2-nitrobenzene (HC Red No. 3), 4-amino-3-nitrophenol, 4-[(2-hydroxyethyl)amino]-3-nitrophenol, 1-amino-5-chloro-4-[(2,3-dihydroxypropyl)amino]-2-nitrobenzene (HC Red No. 10), 5-chloro-1,4-[di(2,3-dihydroxypropyl)-amino]-2-nitrobenzene (HC Red No. 11), 2-chloro-6-ethylamino-4-nitrophenol, 2-amino-6-chloro-4-nitrophenol, 4-[(2-hydroxyethyl)amino]-3-nitro-1-trifluoromethylbenzene (HC Yellow No. 13), 8-amino-2-bromo-5-hydroxy-4-imino-6-{[3-(trimethylammonio)-phenyl]amino}-1(4H)-naphthalenone chloride (C.I. 56059; Basic Blue No. 99), 1-[(4-aminophenyl)azo]-7-(trimethylammonio)-2-naphthol chloride (C.I. 12250; Basic Brown No. 16), 1-[(4-amino-2-nitrophenyl)azo]-7-(trimethylammonio)-2-naphthol chloride (Basic Brown No. 17), 2-hydroxy-1-[(2-methoxyphenyl)azo]-7-(trimethylammonio)naphthalene chloride (C.I. 12245; Basic Red No. 76), 3-methyl-1-phenyl-4-{[3-(trimethylammonio)phenyl]azo}pyrazol-5-one chloride (C.I. 12719; Basic Yellow No. 57) and 2,6-diamino-3-[(pyridin-3-yl)azo]pyridine as well as the salts thereof. In at least one embodiment, the total quantity of direct dyes in the formulation amounts to 0.01 to 15 wt.-%, preferably 0.1 to 10 wt.-%, most preferred 0.5 to 8 wt.-%.

In at least one embodiment, the formulation comprises a conditioning agent. In at least one embodiment, the conditioning agent is a water insoluble, water dispersible, nonvolatile, liquid that forms emulsified, liquid particles. In at least one embodiment, the conditioning agent is a silicone (e.g., silicone oil, cationic silicone, silicone gum, high refractive silicone, or silicone resin), an organic conditioning oil (e.g., hydrocarbon oils, polyolefins, or fatty esters), a cationic conditioning surfactant, a high melting point fatty compound, or combinations thereof.

In at least one embodiment, the conditioning agent is a silicone, and the formulation comprises from 0.01% to 10%, or from 0.1% to 5% silicone conditioning agent, by total weight of the formulation. Suitable silicone conditioning agents, and optional suspending agents for the silicone, are described in U.S. Pat. No. 5,104,646. In at least one embodiment, the formulation comprises a silicone gum selected from the group consisting of polydimethylsiloxane, (polydimethylsiloxane) (methylvinylsiloxane) copolymer, poly(dimethylsiloxane) (diphenylsiloxane) (methylvinylsiloxane) copolymer, and mixtures thereof.

In at least one embodiment, the formulation comprises a terminal aminosilicone. “Terminal aminosilicone” as defined herein means silicone comprising one or more amino groups at one or both ends of the silicone backbone. In at least one embodiment, the formulation is substantially free of any silicone compound comprising pendant amino groups. In an embodiment, the formulation is substantially free of any silicone compound other than terminal aminosilicones. In at least one embodiment, the amino group at at least one terminus of the silicone backbone of the terminal aminosilicone is selected from the group consisting of primary amines, secondary amines and tertiary amines. In at least one embodiment, the formulation comprises from 0.1% to 20%, or from 0.5% to 10%, or from 1% to 6% terminal aminosilicone, by total weight of the formulation.

In at least one embodiment, the formulation comprises a cationic conditioning surfactant. In at least one embodiment, the formulation comprises from 0.05% to 3.0%, or from 0.075% to 2.0%, or from 0.1% to 1.0%, of cationic conditioning surfactant by total weight of the formulation. In at least one embodiment, the cationic conditioning surfactant is comprised in a lamellar gel matrix. In other words, the formulation comprises a lamellar gel matrix and the lamellar gel matrix comprises the cationic conditioning surfactant. In an embodiment, cationic conditioning surfactant is according to Formula (C):

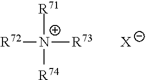

wherein at least one of R⁷¹, R⁷², R⁷³ and R⁷⁴ is selected from an aliphatic group of from 8 to 30 carbon atoms, an aromatic, alkoxy, polyoxyalkylene, alkylamido, hydroxyalkyl, aryl, or an alkylaryl group having up to 22 carbon atoms; the remainder of R⁷¹, R⁷², R⁷³ and R⁷⁴ are independently selected from the group consisting of an aliphatic group of from 1 to 22 carbon atoms, and an aromatic, alkoxy, polyoxyalkylene, alkylamido, hydroxyalkyl, aryl or alkylaryl group having up to 22 carbon atoms; X is selected from the group consisting of halogen, acetate, citrate, lactate, glycolate, phosphate, nitrate, sulfonate, sulfate, alkylsulfate, alkyl sulfonate, and combinations thereof.

In at least one embodiment, the cationic conditioning surfactant is selected from the group consisting of behenyl trimethyl ammonium chloride, methyl sulfate or ethyl sulfate, and stearyl trimethyl ammonium chloride, methyl sulfate or ethyl sulfate. It is believed that a longer alkyl group provides improved smoothness and soft feeling on wet and dry hair, compared to cationic surfactants with a shorter alkyl group. It is also believed that such cationic surfactants can provide reduced irritation, compared to those having a shorter alkyl group.

In at least one embodiment, the cationic surfactant is a di-long alkyl quaternized ammonium salt selected from the group consisting of dialkyl (C14-C18) dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, and mixtures thereof.

In at least one embodiment, the cationic surfactant is a tertiary amido amine having an alkyl group of from 12 to 22 carbons. The tertiary amido amine may be selected from the group consisting of stearamidopropyldimethyl-, stearamidopropyldiethyl-, stearamidoethyldiethyl-, stearamidoethyldimethyl-, palmitamidopropyldimethyl-, palmitamidopropyldiethyl-, palmitamidoethyldiethyl-, palmitamidoethyldimethyl-, behenamidopropyldimethyl-, behenamidopropyldiethyl-, behenamidoethyldiethyl-, behenamidoethyldimethyl-, arachidamidopropyldimethyl-, arachidamidopropyldiethyl-, arachidamidoethyldiethyl-, and arachidamidoethyldimethyl-amine, diethylaminoethylstearamide, and mixtures thereof. A tertiary amido amine may be used in combination with an acid. The acid is typically used as a salt-forming anion. In an embodiment, the acid is selected from the group consisting of lactic acid, malic acid, hydrochloric acid, 1-glumatic acid, acetic acid, citric acid, and mixtures thereof.

In at least one embodiment, the cationic surfactant is selected from the group consisting of cetyltrimethylammonium chloride (CTAC), stearyltrimethylammonium chloride (STAC), behentrimethylammonium methosulfate, stearoylamidopropyldimethyl amine (SAPDMA), distearyldimethylammonium chloride, and mixtures thereof.

In at least one embodiment, the formulation comprises a surfactant system. In at least one embodiment, the surfactant system comprises a surfactant selected from the group consisting of anionic surfactants, cationic surfactants, non-ionic surfactants, zwitterionic surfactants and/or amphoteric surfactants. In at least one embodiment, the formulation comprises a total amount of surfactant of from 0.01 wt.-% to 70 wt.-%, from 0.1 wt.-% to 40%, from 1 wt.-% to 30%, from 2 wt.-% to 20 wt.-%.

In at least one embodiment, the formulation comprises an anionic surfactant. In at least one embodiment, the anionic surfactant is selected from the group consisting of (C₁₀-C₂₀)-alkyl and alkylene carboxylates, alkyl ether carboxylates, fatty alcohol sulfates, fatty alcohol ether sulfates, alkylamide sulfates and sulfonates, fatty acid alkylamide polyglycol ether sulfates, alkanesulfonates and hydroxyalkanesulfonates, olefinsulfonates, acyl esters of isethionates, alpha-sulfo fatty acid esters, alkylbenzenesulfonates, alkylphenol glycol ether sulfonates, sulfosuccinates, sulfosuccinic monoesters and diesters, fatty alcohol ether phosphates, protein/fatty acid condensation products, alkyl monoglyceride sulfates and sulfonates, alkylglyceride ether sulfonates, fatty acid methyltaurides, fatty acid sarcosinates, sulforicinoleates, acylglutamates, and mixtures thereof. The anionic surfactants (and their mixtures) can be used in the form of their water-soluble or water-dispersible salts, examples being the sodium, potassium, magnesium, ammonium, mono, di-, and triethanolammonium, and analogous alkylammonium salts. In at least one embodiment, the anionic surfactant is the salt of an anionic surfactant comprising 12 to 14 carbon atoms. In at least one embodiment, the anionic surfactant is selected from the group consisting of sodium lauryl sulfate, sodium laureth sulfate, sodium tridecyl sulfate, sodium trideceth sulfate, sodium myristyl sulfate, sodium myreth sulfate, and mixtures thereof.

In at least one embodiment, the formulation comprises an acylglycinate surfactant. In at least one embodiment, the acylglycinate surfactant conforms to the formula (Y):

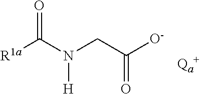

wherein
R^{1a} is a linear or branched, saturated alkyl group having 6 to 30, preferably 8 to 22, particularly preferably 8 to 18 carbon atoms or is a linear or branched, mono- or polyunsaturated alkenyl group having 6 to 30, preferably 8 to 22, particularly preferably 12 to 18 carbon atoms, and
Qₐ⁺ is a cation.

In at least one embodiment, Qₐ⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof. In at least one embodiment, the acylglycinate surfactant is selected from sodium cocoylglycinate and potassium cocoylglycinate. In at least one embodiment, the acylglycinate surfactant is selected from those conforming to formula (Y), wherein R is C12 alkyl or C14 alkyl. In at least one embodiment, the acylglycinate surfactant is selected from those conforming to formula (Y), wherein R is C₁₆ alkyl or C₁₈ alkyl.

In at least one embodiment, the formulation comprises a glutamate surfactant corresponding to formula (Z) or a salt thereof:

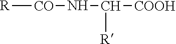

wherein
R′ is HOOC—CH2-CH2- or M⁺⁻OOC—CH2-CH2- wherein M⁺ is a cation;
R is a linear or branched, saturated alkyl group having 6 to 30, preferably 8 to 22, more preferably 8 to 18 carbon atoms or is a linear or branched, mono- or polyunsaturated alkenyl group having 6 to 30, preferably 8 to 22, more preferably 12 to 18 carbon atoms. In at least one embodiment, M⁺ is a metal cation. In at least one embodiment, M⁺ is selected from the group consisting of Li⁺, Na⁺, K⁺, Mg⁺⁺, Ca⁺⁺, Al⁺⁺⁺, NH4⁺, a monoalkylammmonium ion, a dialkylammonium ion, a trialkylammonium ion and a tetraalkylammonium ion, or combinations thereof.

In at least one embodiment, the glutamate surfactant is selected from sodium cocoyl glutamate and potassium cocoyl glutamate. In at least one embodiment, the glutamate surfactant is selected from those conforming to formula (Z), wherein R is C12 alkyl or C14 alkyl. In at least one embodiment, the glutamate surfactant is selected from those conforming to formula (Z), wherein R is C₁₆ alkyl or C₁₈ alkyl.

In at least one embodiment, the formulation comprises from 0.01 wt.-% to 30 wt.-%, preferably from 1 wt.-% to 25 wt.-%, more preferably from 5 wt.-% to 20 wt.-%, particularly preferably from 12 wt.-% to 18 wt.-% anionic surfactant.

In at least one embodiment, the formulation comprises a non-ionic surfactant. In at least one embodiment, the non-ionic surfactant has an HLB (Hydrophilic Lipophilic Balance) of greater than 12. Optionally, the non-ionic surfactant is selected from the group consisting of ethoxylated or ethoxylated/propoxylated fatty alcohols with a fatty chain having 12 to 22 carbon atoms, ethoxylated sterols, such as stearyl- or lauryl alcohol (EO-7), PEG-16 soya sterol or PEG-10 soya sterol, polyoxyethylene polyoxypropylene block polymers (poloxamers), sorbitan esters, sorbitan ester ethoxylates, and mixtures thereof.

In at least one embodiment, the non-ionic surfactant is selected from the group consisting of ethoxylated fatty alcohols, fatty acids, fatty acid glycerides or alkylphenols, in particular addition products of from 2 to 30 mol of ethylene oxide and/or 1 to 5 mol of propylene oxide onto C₈- to C₂₂-fatty alcohols, onto C₁₂- to C₂₂-fatty acids or onto alkyl phenols having 8 to 15 carbon atoms in the alkyl group, C₁₂- to C₂₂-fatty acid mono- and diesters of addition products of from 1 to 30 mol of ethylene oxide onto glycerol, addition products of from 5 to 60 mol of ethylene oxide onto castor oil or onto hydrogenated castor oil, fatty acid sugar esters, in particular esters of sucrose and one or two C₈- to C₂₂-fatty acids, INCI: Sucrose Cocoate, Sucrose Dilaurate, Sucrose Distearate, Sucrose Laurate, Sucrose Myristate, Sucrose Oleate, Sucrose Palmitate, Sucrose Ricinoleate, Sucrose Stearate, esters of sorbitan and one, two or three C₈- to C₂₂-fatty acids and a degree of ethoxylation of from 4 to 20, polyglyceryl fatty acid esters, in particular of one, two or more C₈- to C₂₂-fatty acids and polyglycerol having preferably 2 to 20 glyceryl units, alkyl glucosides, alkyl oligoglucosides and alkyl polyglucosides having C₈ to C₂₂-alkyl groups, e.g. decylglucoside or laurylglucoside, and mixtures thereof.

In at least one embodiment, the non-ionic surfactant is selected from the group consisting of fatty alcohol ethoxylates (alkylpolyethylene glycols), alkylphenol polyethylene glycols, alkylmercaptan polyethylene glycols, fatty amine ethoxylates (alkylaminopolyethylene glycols), fatty acid ethoxylates (acylpolyethylene glycols), polypropylene glycol ethoxylates (e.g. Pluronics®), fatty acid alkylol amides (fatty acid amide polyethylene glycols), N-alkyl-, N-alkoxypolyhydroxy-fatty acid amides, sucrose esters, sorbitol esters, polyglycol ethers, and mixtures thereof.

In at least one embodiment, the formulation comprises a fatty N-methyl-N-glucamide surfactant, wherein the fatty N-methyl-N-glucamide surfactant conforms to the formula (X):

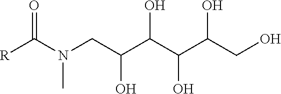

wherein R is selected from saturated or unsaturated hydrocarbon chains having 5 to 23 carbon atoms. Preferably, R in formula (X) is selected from saturated or unsaturated hydrocarbon chains having 7 to 17 carbon atoms. Also preferably, the R—C═O residue in formula (X) is derived from caprylic acid, capric acid, lauric acid, myristic acid, palmitic acid, stearic acid, oleic acid, linoleic acid, linolenic acid, coconut fatty acids, or mixtures thereof. Also preferably, the R—C═O residue in formula (X) is derived from 9-decenoic acid, 9-dodecenoic acid, or mixtures thereof.

Particularly preferred N-methyl-N-acylglucamines of formula (X) are capryloyl/caproyl methyl glucamide, lauroyl/myristoyl methyl glucamide, cocoyl methyl glucamide, oleyl methyl glucamide, or mixtures thereof. Such N-methyl-N-acylglucamines are commercially available from Clariant (GlucoTain® Clear, GlucoTain® Plus, GlucoTain® Flex, GlucoTain® Care, GlucoTain® Sense).

Also particularly preferred N-methyl-N-acylglucamines of formula (X) are N-9-decenoyl-N-methylglucamine, N-9-dodecenoyl-N-methylglucamine, or mixtures thereof.

In at least one embodiment, the formulation comprises from 1 wt.-% to 20 wt.-%, preferably from 2 wt.-% to 10 wt.-%, more preferably from 3 wt.-% to 7 wt.-% non-ionic surfactant.

In at least one embodiment, the formulation comprises an amphoteric surfactant. In at least one embodiment, the amphoteric surfactant is selected from the group consisting of N—(C₁₂-C₁₈)-alkyl-beta-aminopropionates and N—(C₁₂-C₁₈)-alkyl-beta-iminodipropionates as alkali metal salts and mono-, di-, and trialkylammonium salts; N-acylaminoalkyl-N,N-dimethylacetobetaine, preferably N—(C₈-C₁₈)-acylaminopropyl-N,N-dimethylacetobetaine, (C₁₂-C₁₈)-alkyl-dimethyl-sulfopropylbetaine, amphosurfactants based on imidazoline (e.g. Miranol®, Steinapon®), preferably the sodium salt of 1-(beta-carboxymethyloxyethyl)-1-(carboxymethyl)-2-laurylimidazolinium; amine oxides, e.g. (C₁₂-C₁₈)-alkyldimethylamine oxides, fatty acid amidoalkyldimethylamine oxides, and mixtures thereof.

In at least one embodiment, the formulation comprises a betaine surfactant. Optionally, the betaine surfactant is selected from C₈- to C₁₈-alkylbetaines. In at least one embodiment, the betaine surfactant is selected from the group consisting of cocodimethylcarboxymethylbetaine, lauryldimethylcarboxymethylbetaine, lauryldimethylalphacarboxyethylbetaine, cetyldimethylcarboxymethylbetaine, oleyldimethylgammacarboxypropylbetaine and laurylbis(2-hydroxypropyl)alphacarboxyethylbetaine, and combinations thereof. Optionally, the betaine surfactant is selected from C₈- to C₁₈-sulfobetaines. In at least one embodiment, the betaine surfactant is selected from the group consisting of cocodimethylsulfopropylbetaine, stearyldimethylsulfopropylbetaine, lauryldimethylsulfoethylbetaine, laurylbis(2-hydroxyethyl)sulfopropylbetaine, and combinations thereof. Optionally, the betaine surfactant is selected from carboxyl derivatives of imidazole, the C₈- to C₁₈-alkyldimethylammonium acetates, the C₈- to C₁₈-alkyldimethylcarbonylmethylammonium salts, and the C₈- to C₁₈-fatty acid alkylamidobetaines, and mixtures thereof. Optionally, the C₈- to C₁₈-fatty acid alkylamidobetaine is selected from coconut fatty acid amidopropylbetaine, N-coconut fatty acid amidoethyl-N-[2-(carboxymethoxy)ethyl]glycerol (CTFA name: Cocoamphocarboxyglycinate), and mixtures thereof.

In at least one embodiment, the formulation comprises from 0.5 wt.-% to 20 wt.-%, preferably from 1 wt.-% to 10 wt.-% amphoteric surfactant.

In at least one embodiment, the formulation comprises a surfactant system. In at least one embodiment, the surfactant system comprises at least one surfactant selected from the group consisting of lauryl sulfate, laureth sulfate, cocoamido-propylbetaine, sodium cocoylglutamate, lauroamphoacetate, and mixtures thereof. In at least one embodiment, the surfactant system comprises sodium laureth sulphate, sodium lauryl sulphate, and optionally cocamidopropyl betaine. In at least one embodiment, the surfactant system comprises sodium laureth sulphate, potassium cocoyl glutamate, and cocamidopropyl betaine.

In at least one embodiment, the formulation has a viscosity (at 25 °C) of from 0.1 cPs to 20,000 cPs. In at least one embodiment, the formulation has a viscosity (at 25 °C) of from 0.1 cPs to 10,000 cPs, or from 1 cPs to 5,000 cPs, or from 5 cPs to 3,500 cPs. Viscosity may be important for anti-drip reasons. Dripping can be inconvenient for the user. Furthermore, more viscous formulations can be useful for measured dispensing. In at least one embodiment, the formulation has a viscosity (at 25 °C) of from 0.1 cPs to 1,000 cPs. This viscosity range is advantageous when the formulation is in the form of a facial cleanser in view of the need for distribution on skin and ability to rinse off.

In at least one embodiment, the formulation further comprises a viscosity-modifying substance. The viscosity-modifying substance is preferably a thickening polymer.

In at least one embodiment, the thickening polymer is a polymer based on acrylamidomethylpropanesulfonic acid (AMPS®). These polymers, even at pH values of 7 or less, exhibit good thickening performance. Especially preferably, the thickening polymer is selected from the group consisting of homo- or copolymers of acrylamidomethylpropanesulfonic acid and salts thereof. Among the polymers just mentioned, preference is given to polymers having at least 20 mol-% of units based on acrylamidomethylpropanesulfonic acid and/or salts thereof, and particular preference to polymers having at least 50 mol-% of units based on acrylamidomethylpropanesulfonic acid and/or salts thereof, the mole figures relating in each case to the overall polymer. In the case of the copolymers, in addition to structural units based on acrylamidomethylpropanesulfonic acid and/or salts thereof, preferably one or more structural units based on the following comonomers are present in the copolymers: acrylic acid, methacrylic acid, acrylamide, dimethylacrylamide, vinylpyrrolidone (VP), hydroxyethyl acrylate, hydroxyethyl methacrylate, acrylic or methacrylic esters of ethoxylated alcohols RO—(CH₂CH₂O)ₘH, in which R is an alkyl radical having 12 to 30 carbon atoms and m is a number from 3 to 30, and CH₂═CH—COO—(CH₂CH₂—COO)ₙX, in which n is a number from 0 to 10 and X is a counterion and is preferably H⁺, Na⁺ and/or NH4⁺. The polymers selected from the group consisting of homo- or copolymers of acrylamidomethylpropanesulfonic acid and salts thereof may be crosslinked or non-crosslinked. In the case of crosslinking, they contain structural units based on monomers having 2 or more olefinic double bonds. In the case of crosslinking, preferably from 0.1 to 10 mol-% of such structural units are present in the homo- or copolymers, based on the overall polymer. If one or more structural units based on acrylamidomethylpropanesulfonic acid and/or salts thereof in the homo- or copolymers of acrylamidomethylpropanesulfonic acid and/or salts thereof have one or more counterions other than H⁺, these other counterions are preferably selected from the group consisting of Na⁺ and NH4⁺. Suitable polymers are mentioned in publications including EP-0816403, EP-1069142, EP-1116733 and DE-10 2009 014877 (Clariant), EP-1347736 (L'Oréal) or EP-1496081 (Seppic). Examples include: Aristoflex® AVC (Ammonium Acryloyldimethyltaurate/VP Copolymer), Aristoflex® AVS (Sodium Acryloyldimethyltaurate/VP Crosspolymer), Aristoflex® TAC (Ammonium Acryloyl Dimethyltaurate Carboxyethyl Acrylate Crosspolymer), Hostacerin® AMPS (Ammonium Polyacryloyldimethyl Taurate), Aristoflex® HMB (Ammonium Acryloyldimethyltaurate/Beheneth-25 Methacrylate Crosspolymer), Aristoflex® BLV (Ammonium Acryloyldimethyltaurate/Beheneth-25 Methacrylate Crosspolymer), Aristoflex® HMS (Ammonium Acryloyldimethyltaurate/Steareth-25 Methacrylate Crosspolymer), Aristoflex® SNC (Ammonium Acryloyldimethyltaurate/Steareth-8 Methacrylate Copolymer), Aristoflex® LNC (Ammonium Acryloyldimethyltaurate/Laureth-7 Methacrylate Copolymer) or Sepinov® EMT 10 (Hydroxyethyl Acrylate/Sodium Acryloyldimethyl Taurate Copolymer), Sepigel® 305.

In at least one embodiment, the thickening polymer is selected from the group consisting of copolymers of at least one first monomer type, which is chosen from acrylic acid and methacrylic acid, and at least one second monomer type, which is chosen from esters of acrylic acid and ethoxylated fatty alcohol; crosslinked polyacrylic acid; crosslinked copolymers of at least one first monomer type, which is chosen from acrylic acid and methacrylic acid, and at least one second monomer type, which is chosen from esters of acrylic acid with C₁₀- to C₃₀-alcohols; copolymers of at least one first monomer type, which is chosen from acrylic acid and methacrylic acid, and at least one second monomer type, which is chosen from esters of itaconic acid and ethoxylated fatty alcohol; copolymers of at least one first monomer type, which is chosen from acrylic acid and methacrylic acid, at least one second monomer type, which is chosen from esters of itaconic acid and ethoxylated C₁₀- to C₃₀-alcohols, and a third monomer type, which is chosen from C₁- to C₄-aminoalkyl acrylates; copolymers of two or more monomers chosen from acrylic acid, methacrylic acid, acrylic esters and methacrylic esters; copolymers of vinylpyrrolidone and ammonium acryloyldimethyltaurate; copolymers of ammonium acryloyldimethyltaurate and monomers chosen from esters of methacrylic acid and ethoxylated fatty alcohols; hydroxyethylcellulose; hydroxypropylcellulose; hydroxypropylguar; glyceryl polyacrylate; glyceryl polymethacrylate; copolymers of at least one C₂-, C₃- or C₄-alkylene and styrene; polyurethanes; hydroxypropyl starch phosphate; polyacrylamide; copolymers of maleic anhydride and methyl vinyl ether crosslinked with decadiene; carob seed flour; guar gum; xanthan; dehydroxanthan; carrageenan; karaya gum; hydrolyzed corn starch; copolymers of polyethylene oxide, fatty alcohols and saturated methylenediphenyl diisocyanate (e.g. PEG-150/stearyl alcohol/SMDI copolymer); and mixtures thereof.

In at least one embodiment, the formulation has a pH value of from 2.0 to 12.0, preferably from 3.0 to 9.0, more preferably from 4.5 to 7.5. By varying the pH value, a formulation can be made available that is suitable for different applications.

In at least one embodiment, the formulation comprises an alkalizing agent or pH adjusting agent. In at least one embodiment, ammonia or caustic soda is suitable, but water-soluble, physiologically tolerable salts of organic or inorganic bases can also be considered. Optionally, the pH adjusting agent is selected from ammonium hydrogen carbonate, ammonia, monoethanolamine, ammonium carbonate. In at least one embodiment, the alkalizing agent or pH adjusting agent is selected from the group consisting of 2-amino-2-methyl-1-propanol, 2-amino-2-methyl-1,3-propanediol, 2-amino-2-ethyl-1,3-propanediol, tris(hydroxymethyl)-aminomethane, 2-amino-1-butanol, tris(2-hydroxypropyl)-amine, 2,2-iminobisethanol, lysine, iminourea (guanidine carbonate), tetrahydro-1,4-oxazine, 2-amino-5-guanidin-valeric acid, 2-aminoethansulfonic acid, diethanolamine, triethanolamine, N-methyl ethanolamine, isopropanolamine, diisopropanolamine, triisopropanolamine, glucamine, sodium hydroxide, potassium hydroxide, lithium hydroxide, magnesium oxide, and mixtures thereof.

To establish an acidic pH value, an acid can be included. In at least one embodiment, the formulation comprises an acid selected from the group consisting of hydrochloric acid, phosphoric acid, acetic acid, formic acid, sulfuric acid, citric acid, and mixtures thereof. Citric acid is most preferred in that it has high consumer acceptance. In at least one embodiment, the acidic pH is adjusted with a buffer such as a phosphate buffer, a TRIS buffer or a citric buffer. The buffers may be used alone or in combination with an acid.

The formulation of the present invention may be prepared by methods known in the art. For example, the formulation may be prepared by mixing its ingredients, preferably at ambient temperature (20 °C) and ambient pressure (1013 mbar).

The examples which follow are intended to illustrate the subject matter of the invention without restricting it thereto.

### EXAMPLES

### Example 1

| | | | Hy- | PEG- | | | |
|---|---|---|---|---|---|---|---|
| | Propy- | | droxy- | 40 | Ascor- | | |
| | lene | Leci- | ethyl | Mono- | bic | Wa- | To- |
| Ex- | Glycol | thin | Urea | stearate | Acid | ter | tal |
| am- | (% | (% | (% | (% | (% | (% | (% |
| ple | w/w) | w/w) | w/w) | w/w) | w/w) | w/w) | w/w) |
| 1-1 | 12.9 | 2.6 | 13.0 | 12.9 | 10.0 | 48.6 | 100 |
| 1-2 | 14.2 | 11.3 | 17.0 | 11.3 | 10.6 | 35.6 | 100 |
| 1-3 | 13.5 | 5.4 | 10.8 | 16.2 | 8.5 | 45.6 | 100 |
| 1-4 | 8.6 | 11.4 | 11.4 | 11.4 | 9.0 | 48.2 | 100 |
| 1-5 | 0 | 0 | 0 | 0 | 10 | 90 | 100 |

### Preparation of Ascorbic Acid Solution:

Step 1. Weigh propylene glycol (PG) into a container and heat it to 75-90 °C
Step 2. Weigh phospholipid and add into the heated PG and stir using an overhead stirrer at 75-90 °C until a clear homogeneous solution is obtained
Step 3. Weigh PEG-40 monostearate and add into the transparent solution obtained in Step 2 and stir using an overhead stirrer at 75-90 °C until a clear homogeneous solution is obtained
Step 4. Weigh hydroxyethyl urea and add into the transparent solution obtained in Step 3 and stir using an overhead stirrer at 75-90 °C until a clear homogeneous solution is obtained
Step 5. Weigh ascorbic acid and add in the transparent solution obtained in step 4 and stir using an overhead stirrer at 75-90 °C until a clear homogeneous solution is obtained
Step 6. Cool down the temperature of the transparent solution obtained in Step 5 to ambient temperature to obtain the ascorbic acid solution

### Preparation of Aqueous Ascorbic Acid System:

Step 1. Weigh ascorbic acid solution into a beaker and stir gently using an overhead stirrer at ambient temperature
Step 2. Weigh water into a dropping funnel keeping the stopper closed and add water dropwise into the ascorbic acid solution from the dropping funnel while gently stirring under overhead stirrer until a homogeneous mixture was obtained
Step 3. Store the aqueous ascorbic acid system under standard storage conditions

### Measurement of Ascorbic Acid:

### 1% Starch Indicator Solution:

Make a paste of 0.50 g soluble starch in little water and then add to 50 ml near boiling distilled water, while stirring. Stir for 1-5 minutes to obtain a homogeneous solution
Allow to cool, add 0.1-0.5 mg of potassium iodide

### Determination of Ascorbic Acid in Sample:

Take 10 ml of standard ascorbic acid solution in a conical flask
Add 2-3 drops of starch indicator solution
Titrate the solution with iodine solution and note down the volume (V₁)
Now take 10 ml solution of unknown ascorbic acid concentration
Add 2-3 drops of starch indicator solution
Titrate the solution with iodine solution and note down the volume (V₂)

### Calculation of Ascorbic Acid Concentration of Ascorbic Acid Sample (S₂):

S₂=V₂×S₁/V₁

### Where

S₁ is the concentration of standard ascorbic acid solution (which is 0.1% in this case)
V₁: volume of iodine solution required for titrating 10 ml of standard ascorbic acid solution
V₂: volume of iodine solution required for titrating 10 ml of the sample (solution of unknown ascorbic acid concentration)

### Results

| | % Ascorbic | % Ascorbic Acid* |
|---|---|---|
| | Acid* (after | (after 4 weeks |
| Example | 4 weeks @ RT) | @ 45 °C) |
| 1-1 | 100 | 90.3 |
| 1-2 | 94.0 | 90.5 |
| 1-3 | 100 | 95.2 |
| 1-4 | 98.9 | 92.1 |
| 1-5 | 74.2 | 70.1 |

*based on the initial amount of ascorbic acid, i.e. at time 0

### Example 2

| | | | Hy- | PEG- | | | |
|---|---|---|---|---|---|---|---|
| | Propy- | | droxy- | 40 | Nia- | | |
| | lene | Leci- | ethyl | Mono- | cin- | Wa- | To- |
| Ex- | Glycol | thin | Urea | stearate | amide | ter | tal |
| am- | (% | (% | (% | (% | (% | (% | (% |
| ple | w/w) | w/w) | w/w) | w/w) | w/w) | w/w) | w/w) |
| 2-1 | 12.9 | 2.0 | 13.3 | 13.3 | 10 | 48.5 | 100 |
| 2-2 | 12.9 | 3.0 | 12.8 | 12.8 | 10 | 48.5 | 100 |
| 2-3 | 12.9 | 2.0 | 10.8 | 10.8 | 15 | 48.5 | 100 |
| 2-4 | 12.9 | 2.0 | 8.3 | 8.3 | 20 | 48.5 | 100 |
| 2-5 | 0.0 | 0.0 | 0.0 | 0.0 | 10 | 90 | 100 |
| 2-6 | 0.0 | 0.0 | 0.0 | 0.0 | 15 | 85 | 100 |
| 2-7 | 0.0 | 0.0 | 0.0 | 0.0 | 20 | 80 | 100 |

### Preparation of Niacinamide Solution:

Step 1. Weigh propylene glycol (PG) into a container and heat it to 75-90 °C
Step 2. Weigh phospholipid and add into the heated PG and stir using an overhead stirrer at 75-90 °C until a clear homogeneous solution is obtained
Step 3. Weigh PEG-40 monostearate and add into the transparent solution obtained in Step 2 and stir using an overhead stirrer at 75-90 °C until a clear homogeneous solution is obtained
Step 4. Weigh hydroxyethyl urea and add into the transparent solution obtained in Step 3 and stir using an overhead stirrer at 75-90 °C until a clear homogeneous solution is obtained
Step 5. Weigh niacinamide and add in the transparent solution obtained in step 4 and stir using an overhead stirrer at 75-90 °C until a clear homogeneous solution is obtained
Step 6. Cool down the temperature of the transparent solution obtained in Step 5 to 45 °C to obtain the niacinamide solution

### Preparation of Aqueous Niacinamide System:

Step 1. Weigh niacinamide solution into a beaker and stir gently using an overhead stirrer at 45 °C
Step 2. Weigh water and set the temperature to 45 °C. Add water dropwise into the niacinamide solution gently stirring under overhead stirrer until a homogeneous mixture was obtained
Step 3. Store the aqueous niacinamide system under standard storage conditions

### Measurement of Niacinamide:

The niacinamide content in the formulation is quantitatively analyzed using HPLC.

### Results

| | % Niacinamide* | % Niacinamide* |
|---|---|---|
| | (after 4 weeks | (after 4 weeks |
| Example | @ RT) | @ 40 °C) |
| 2-1 | 100 | 100 |
| 2-2 | 100 | 100 |
| 2-3 | 98.6 | 100 |
| 2-4 | 100 | 100 |

*based on the initial amount of niacinamide, i.e. at time 0

In conclusion, the niacinamide compositions of the present invention are stable.

### HPLC Method:

1. Instrument
Method: Quantification by HPLC—DAD/UV
Equipment: Agilent 1260 Infinity-II HPLC
Column: Agilent Eclipse plus C-18, length 150 mm, ID 4.6 mm×3.5 μm (S/N USUXCO7215)
Wavelength: 260±4 nm
Flow: 0.5 ml/min (Gradient flow)
Temperature: 40 °C
Injection volume: 5 μl
Run time: 18 mins
Mobile Phase:
Eluent-1: 0.005 mol/1 Tetrabutylammonium hydrogen sulphate: Acetonitrile (95:5)% v/v
Eluent-2: Acetonitrile (100%)

### Gradient:

| Time (mins) | % Eluent -1 | % Eluent -2 | Flow (ml) |
|---|---|---|---|
| 0 | 98 | 2 | 0.5 |
| 5 | 98 | 2 | 0.5 |
| 8 | 50 | 50 | 0.5 |
| 10 | 10 | 90 | 0.5 |
| 15 | 10 | 90 | 0.5 |
| 16 | 98 | 2 | 0.5 |
| 18 | 98 | 2 | 0.5 |

2. Procedure
   Inject diluent
   Inject standard solution (100 ppm) twice
   Inject diluent
   Inject test solution
   Inject diluent
3. Diluent solution: Acetonitrile:water (70:30) v/v
4. Standard solution
Preparation of standard stock solution (1000 ppm):
Weigh 25±10 mg of niacinamide in 25 ml volumetric flask and add 20 ml water.
Sonicate to dissolve and dilute up to the mark with water.
Preparation of standard solution (100 ppm):
Further dilute 1 ml of stock solution to 10 ml with diluent.
5. Test solution
Weigh 1000.0±5.0 mg of sample in 100 ml volumetric flask. Then add 50 ml water, dissolve and dilute up to the mark with water. Further pipette out 1 ml of the solution and dilute up to 10 ml with diluent.
6. Evaluation

$$\% Niacinamide content = \frac{Area of test \times conc of standard}{Area of standard \times conc of test} \times Purity of standard$$

### Example 3

Incorporation of the aqueous niacinamide system into a facial serum chassis: Add a niacinamide composition of Example 2 to the following facial serum chassis composition SC1. The mixing ratio is chosen such that the final concentration of niacinamide in the facial serum chassis is 5%. Adjust the pH of all the samples to pH 3. Store the samples at room temperature and at 40 °C.

### Facial Serum Chassis Composition SC1:

| Ingredient | wt % |
|---|---|
| Aqua | 90 |
| Tween 20 | 1.0 |
| Glycerin | 4.0 |
| Xanthan Gum | 0.50 |
| Phenoxyethanol | 0.25 |
| DMDM Hydantoin/Sodium Benzoate | 0.25 |
| Water | q.s. to |
| | 100 |

### Measurement of Niacinamide:

The niacinamide content in the formulation is quantitatively analyzed using HPLC as described in Example 2.

### Results

| | Niacinamide | % | % |
|---|---|---|---|
| | composition | Niacinamide* | Niacinamide* |
| | of | (after 4 weeks | (after 4 weeks |
| Example | Example | @ RT) | @ 40 °C) |
| 3-1 | 2-1 | 100 | 89.4 |
| 3-2 | 2-2 | 100 | 93.3 |
| 3-3 | 2-3 | 100 | 100 |
| 3-4 | 2-4 | 100 | 100 |

*based on the initial amount of niacinamide, i.e. at time 0

In conclusion, the niacinamide content in the facial serum chassis containing the niacinamide composition of the present invention is stable.

### Example 4

### Release Study of the Niacinamide Compositions of Example 2

### Mechanism:

Niacinamide released from the samples will diffuse through the dialysis membrane to the outer compartment (buffer solution)

Steps involved to determine the release percentage of niacinamide:
Tie one end of the dialysis tube with thread
Add 1 ml of the sample into the tube and tie the top with thread
Add 250 ml of pH 5.5 buffer solution into a beaker
Place the dialysis tube containing the sample in the beaker
Stir the buffer solution using magnetic stirrer
Collect 1 ml aliquots (dialysate) at different intervals
Analyze the collected aliquots (dialysate) using HPLC as described in Example 2

### Example 4a

Release study of niacinamide compositions having a niacinamide content of 10%. The following Table indicates the release of niacinamide in % at a given time, based on the initial amount of niacinamide, i.e. at time 0:

| | Niacinamide | Niacinamide | Niacinamide |
|---|---|---|---|
| | composition of | composition of | composition of |
| Time (hr) | Example 2-1 | Example 2-2 | Example 2-5 |
| 0 | 0.0 | 0.0 | 0.0 |
| 0.08 | 5.8 | 5.4 | 16.3 |
| 0.17 | 10.4 | 9.7 | 26.2 |
| 0.25 | 15.0 | 14.3 | 35.7 |
| 0.5 | 27.7 | 23.4 | 55.1 |
| 1 | 45.9 | 46.0 | 80.9 |
| 2 | 63.6 | 59.8 | 90.8 |
| 3 | 67.2 | 63.3 | 92.5 |
| 4 | 67.9 | 64.4 | 92.8 |
| 5 | 68.3 | 65.3 | 92.6 |
| 6 | 68.2 | 67.8 | 92.8 |

In conclusion, the niacinamide compositions of the present invention release niacinamide more slowly than the composition of the comparative example.

### Example 4b

### Release Study of Niacinamide Compositions Having a Niacinamide Content of 15%.

The following Table indicates the release of niacinamide in % at a given time, based on the initial amount of niacinamide, i.e. at time 0:

| | Niacinamide | Niacinamide |
|---|---|---|
| | composition of | composition of |
| Time (hr) | Example 2-3 | Example 2-6 |
| 0.00 | 0 | 0.0 |
| 0.08 | 4.8 | 12.8 |
| 0.17 | 9.2 | 20.7 |
| 0.25 | 13.7 | 28.7 |
| 0.50 | 25.1 | 47.0 |
| 1.00 | 42.8 | 72.1 |
| 2.00 | 58.2 | 85.7 |
| 3.00 | 62.5 | 88.2 |
| 4.00 | 63.9 | 88.2 |
| 5.00 | 64.1 | 88.4 |
| 6.00 | 64.3 | 88.6 |

In conclusion, the niacinamide compositions of the present invention release niacinamide more slowly than the composition of the comparative example.

### Example 4c

### Release Study of Niacinamide Compositions Having a Niacinamide Content of 20%.

The following Table indicates the release of niacinamide in % at a given time, based on the initial amount of niacinamide, i.e. at time 0:

| | Niacinamide | Niacinamide |
|---|---|---|
| Time | composition of | composition of |
| (hr) | Example 2-4 | Example 2-7 |
| 0.00 | 0.0 | 0.0 |
| 0.08 | 9.9 | 6.6 |
| 0.17 | 15.3 | 14.4 |
| 0.25 | 20.9 | 21.8 |
| 0.50 | 36.1 | 39.0 |
| 1.00 | 55.0 | 62.1 |
| 2.00 | 68.4 | 74.2 |
| 3.00 | 71.8 | 75.0 |
| 4.00 | 72.6 | 76.8 |
| 5.00 | 72.8 | 76.8 |
| 6.00 | 72.7 | 76.9 |

In conclusion, the niacinamide compositions of the present invention release niacinamide more slowly than the composition of the comparative example.

### Example 5

### Release Study of the Facial Serum Chassis of Example 3

### Mechanism and Steps Involved to Determine the Release Percentage of Niacinamide: As Described in Example 4

The following Table indicates the release of niacinamide in % at a given time, based on the initial amount of niacinamide, i.e. at time 0:

| | Facial | Facial | Facial | Facial | |
|---|---|---|---|---|---|
| | serum | serum | serum | serum | |
| | chassis | chassis | chassis | chassis | Facial |
| | of | of | of | of | serum |
| Time | Example | Example | Example | Example | chassis |
| (hr) | 3-1 | 3-2 | 3-3 | 3-4 | 3-5* |
| 0.0 | 0.0 | 0.0 | 0.0 | 0.0 | 0.0 |
| 0.1 | 6.8 | 7.9 | 8.4 | 9.3 | 12.2 |
| 0.2 | 11.4 | 11.8 | 12.9 | 14.4 | 18.7 |
| 0.3 | 16.2 | 16.1 | 16.8 | 17.9 | 22.4 |
| 0.5 | 29.1 | 26.5 | 27.4 | 28.4 | 38.7 |
| 1.0 | 49.0 | 43.1 | 43.5 | 50.1 | 62.3 |
| 2.0 | 69.8 | 61.1 | 60.2 | 69.0 | 79.2 |
| 3.0 | 79.8 | 66.3 | 64.9 | 75.7 | 84.1 |
| 4.0 | 82.7 | 68.6 | 65.9 | 78.9 | 87.3 |
| 5.0 | 83.7 | 68.8 | 66.7 | 79.8 | 86.2 |
| 6.0 | 83.8 | 69.0 | 66.8 | 79.9 | 86.6 |

*Facial serum chassis 3-5 was prepared as described in Example 3 using the niacinamide composition of Example 2-5

In conclusion, the facial serum chassis containing the niacinamide composition of the present invention release niacinamide more slowly than the facial serum chassis of the comparative example.

### Example 6

The vitamin compositions of Examples 1 and 2 can be incorporated into a serum chassis. Any of the vitamin compositions of Examples 1 and 2 can be mixed with the following serum chassis composition SC2. The mixing ratio can be chosen such that the final concentration of niacinamide in the serum chassis is 5%. The pH of the resulting formulations can be adjusted to pH 3, pH 5 or pH 7.

### Serum Chassis Composition SC2:

| Ingredient | wt % |
|---|---|
| Water | 91.7 |
| Butylene glycol | 4.0 |
| Allantoin | 0.5 |
| Sorbitol | 1.0 |
| Capryloyl/Caproyl Anhydro Methyl Glucamide | 1.5 |
| Phenoxyethanol | 0.5 |
| Sodium Polyacryloyldimethyl Taurate | 0.8 |
| Sodium hydroxide | q.s |
| Citric acid | q.s |

### Example 7

The following aqueous niacinamide systems (Examples 7-1 and 7-2) were prepared as described in Example 2.

| | | | Hy- | PEG- | | | |
|---|---|---|---|---|---|---|---|
| | Propy- | | droxy- | 40 | Nia- | | |
| | lene | Leci- | ethyl | Mono- | cin- | Wa- | To- |
| Ex- | Glycol | thin | Urea | stearate | amide | ter | tal |
| am- | (% | (% | (% | (% | (% | (% | (% |
| ple | w/w) | w/w) | w/w) | w/w) | w/w) | w/w) | w/w) |
| 7-1 | 12.9 | 2.0 | 13.3 | 13.3 | 20 | 38.5 | 100 |
| 7-2 | 12.9 | 3.0 | 12.8 | 12.8 | 20 | 38.5 | 100 |

The niacinamide content was measured as described in Example 2. Results:

| | % Niacinamide* | % Niacinamide* |
|---|---|---|
| | (after 4 weeks | (after 4 weeks |
| Example | @ RT) | @ 40 °C) |
| 7-1 | 100 | 100 |
| 7-2 | 100 | 100 |

*based on the initial amount of niacinamide, i.e at time 0

In conclusion, the niacinamide compositions of the present invention are stable.

1-16. (canceled)

17. A vitamin composition comprising
(a) a vitamin or derivative thereof;
(b) a lipid component;
(c) optionally an emulsifier;
(d) a solvent; and
(e) optionally urea or a derivative thereof.

18. The vitamin composition according to claim 17, wherein the vitamin or derivative thereof is present in the vitamin composition in an amount of from 1 to 40 wt-%, based on the total weight of the vitamin composition.

19. The vitamin composition according to claim 17, wherein the vitamin or derivative thereof and the lipid component are present in a weight-to-weight ratio of from 15:1 to 1:2.

20. The vitamin composition according to claim 17, wherein the vitamin or derivative thereof and the solvent are present in a weight-to-weight ratio of from 5:1 to 1:5.

21. The vitamin composition according to claim 17, wherein the vitamin is selected from vitamin A, vitamin B, vitamin C, vitamin D, vitamin E, vitamin K, and mixtures thereof.

22. The vitamin composition according to claim 17, wherein the vitamin or derivative thereof is selected from ascorbic acid, sodium ascorbyl phosphate, magnesium ascorbyl phosphate, ascorbyl glucoside, tetrahexyldecyl ascorbate, ethylated ascorbic acid, 3-O-glycosyl-ascorbic acid, ascorbic acid 2-glucoside, and mixtures thereof.

23. The vitamin composition according to claim 17, wherein the vitamin or derivative thereof is selected from niacin (nicotinic acid), niacinamide (nicotinamide), nicotinamide mononucleotide, nicotinamide riboside, dihydronicotinic acid riboside, 1-methylnicotinamide, inositol hexaniacinate, and mixtures thereof.

24. The vitamin composition according to claim 17, wherein the lipid component is selected from lipids, phospholipids, and mixtures thereof.

25. The vitamin composition according to claim 17, wherein the phospholipids are selected from phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, ceramide phosphorylcholine, ceramide phosphorylethanolamine, ceramide phosphoryllipid, and mixtures thereof.

26. The vitamin composition according to claim 17, wherein the vitamin composition further comprises
(c) an emulsifier selected from fatty alcohols, fatty acids, glyceryl esters, sorbitan esters, sorbitol esters, polyglyceryl esters, sucrose esters, phosphates, fatty alcohol ethoxylates, fatty acid ethoxylates, lactylates, and mixtures thereof.

27. The vitamin composition according to claim 17, wherein the solvent is selected from propylene glycol (1,2-propanediol), 1,3-propanediol, 1,2-butanediol, 1,3-butanediol, 1,2-pentanediol, dipropylene glycol, ethylene glycol, polyethylene glycol, sorbitol, glycerin, and mixtures thereof.

28. The vitamin composition according to claim 17, wherein the vitamin composition further comprises
(e) urea or a derivative thereof selected from 2-hydroxyethyl urea, 2-hydroxymethyl urea, bis(hydroxyethyl) urea, bis(hydroxymethyl) urea, N-(2-hydroxyethyl)ethylene urea, and mixtures thereof.

29. The vitamin composition according to claim 17, wherein the vitamin composition further comprises
(f) water.

30. The vitamin composition according to claim 29, wherein the vitamin or derivative thereof and water are present in a weight-to-weight ratio of from 1:2 to 1:6.

31. A process for the preparation of the vitamin composition according to claim 17, wherein the process comprises:
preparing a mixture comprising a vitamin or derivative thereof, a lipid component, optionally an emulsifier, a solvent, and optionally urea or a derivative thereof; and
optionally adding water to the mixture.

32. An antioxidant, anti-ageing agent, anti-wrinkle agent, skin lightening agent, skin whitening agent, depigmenting agent, anti-inflammatory agent, or anti-acne agent comprising the vitamin composition according to claim 17.

33. A cosmetic formulation comprising
(A) the vitamin composition according to claim 17 in an amount such that the vitamin or derivative thereof is present in an amount of from 1 to 30 wt-%, based on the total weight of the cosmetic formulation; and
(B) one or more further components.

34. The cosmetic formulation according to claim 33, wherein the cosmetic formulation comprises at least 50 wt-% of water, based on the total weight of the cosmetic formulation.

35. A method of treating skin, the method comprising applying to the skin the vitamin composition according to claim 17.
